(19) United States
(12) Patent Application Publication
(10) Pub. No.: US 2012/0108668 A1
(43) Pub. Date: May 3, 2012

(54) METABOLIC PROFILES

(76) Inventors: Erik Torbjörn Lundstedt, Uppsala (SE); Nils Johan Trygg, Umea (SE); Jon Robert Gabrielsson, Umea (SE); Gunilla Ekström, Lund (SE); Nils Johan Trygg, Umea (SE); Jon Robert Gabrielsson, Umea (SE); Gunilla Ekström, Lund (SE)

(21) Appl. No.: 13/259,733

(22) PCT Filed: Mar. 24, 2010
(86) PCT No.: PCT/GB2010/000557

§ 371 Date: Jan. 11, 2012

(30) Foreign Application Priority Data
Mar. 24, 2009 (GB) 0905090.7
Mar. 24, 2009 (GB) 0905096.4
Mar. 24, 2009 (GB) 0905098.0

(51) Int. Cl. A61K 31/155; A61P 19/02

(52) U.S. Cl. 514/632; 514/634

(57) ABSTRACT

The invention relates to the use of endogenous metabolites to produce a metabolic profile of a disorder or disease in a subject, e.g. an autoimmune disease, in particular rheumatoid arthritis, and the analysis of such metabolic profiles in order to find disturbances in such profiles in a subject which are caused by or correlated with the said diseases or disorders. Such disturbances can be normalised by treatment of the subject with specified compounds, particularly N-(2-chloro-3,4-dimethoxybenzylideneamino)guanidine or an aminoguanidine.

The invention relates to the use of endogenous metabolites to produce a metabolic profile of a disorder or disease, in a subject, e.g. an autoimmune disease, in particular rheumatoid arthritis, and the analysis of such metabolic profiles in order to find disturbances in such profiles in a subject which are caused by or correlated with the said diseases or disorders. Such disturbances can be normalised by treatment of the subject with specified compounds, particularly N-(2-chloro-3,4-dimethoxybenzylideneamino)guanidine or an aminoguanidine.

All autoimmune diseases (AD) are caused by failure of the mechanisms that regulate immune system function. In 2005 it was estimated that 5-8% of the US population suffered from an AD (National Institute of Health (2005)). There are more than 80 diseases classified as ADs and a number of diseases are also suspected to be autoimmune, see Table 1 for a list of prevalent ADs together with diseases with suspected autoimmune contributions.

TABLE 1
List of accepted and suspected ADs.

| | | Accepted/ |
|---|---|---|
| Num | Name | Suspected |
| 1 | Acute disseminated encephalomyelitis | Accepted |
| 2 | Addison's disease | Accepted |
| 3 | Alopecia Areata | Accepted |
| 4 | Ankylosing spondylitis | Accepted |
| 5 | Antiphospholipid antibody syndrome | Accepted |
| 6 | Atherosclerosis | Accepted |
| 7 | Autism | Suspected |
| 8 | Autoimmune hepatitis | Accepted |
| 9 | Autoimmune lymphoproliferative syndrome | Accepted |
| 10 | Autoimmune polyendocrine syndromes | Accepted |
| 11 | Behcet's Disease | Accepted |
| 12 | Bell's Palsy | Suspected |
| 13 | Bullous pemphigoid | Accepted |
| 14 | Coeliac disease | Accepted |
| 15 | Chagas' disease | Suspected |
| 16 | Chronic Fatigue Syndrome | Suspected |
| 17 | Chronic obstructive pulmonary disease | Suspected |
| 18 | Crohn's disease | Accepted |
| 19 | Dermatitis herpetiformis | Accepted |
| 20 | Dermatomyositis | Accepted |
| 21 | Diabetes Insipidus | Accepted |
| 22 | Diabetes mellitus type 1 | Accepted |
| 23 | Diabetes mellitus type 2 | Suspected |
| 24 | Discoid lupus erythematosus | Suspected |
| 25 | Glomerulonephritis | Accepted |
| 26 | Goodpasture's syndrome | Accepted |
| 27 | Graves disease | Accepted |
| 28 | Guillain-Barré syndrome (Miller Fisher Syndrome) | Accepted |
| 29 | Hashimoto's Thyroiditis | Accepted |
| 30 | Hemolytic anemia | Accepted |
| 31 | Hemophilia | Accepted |
| 32 | Hidradenitis suppurativa | Suspected |
| 33 | Idiopathic thrombocytopenic purpura | Accepted |
| 34 | IgA nephropathy | Accepted |
| 35 | Mixed connective tissue disease | Accepted |
| 36 | Morphea | Suspected |
| 37 | Multiple sclerosis | Accepted |
| 38 | Myelopathy | Accepted |
| 39 | Osteoarthritis | Suspected |
| 40 | Pemphigus | Accepted |
| 41 | Pernicious anaemia | Accepted |
| 42 | Polymyositis | Accepted |
| 43 | Primary biliary cirrhosis | Accepted |
| 44 | Primary sclerosing cholangitis | Accepted |
| 45 | Psoriasis | Accepted |
| 46 | Psoriatic Arthritis | Accepted |
| 47 | Rasmussen's Encephalitis | Suspected |
| 48 | Relapsing Polychondritis | Accepted |
| 49 | Rheumatoid arthritis | Accepted |
| 50 | Schizophrenia | Suspected |
| 51 | Scleroderma | Accepted |
| 52 | Sjögren's syndrome | Accepted |
| 53 | Systemic lupus erythematosus | Accepted |
| 54 | Temporal arteritis (giant cell arteritis) | Accepted |
| 55 | Thyroiditis | Accepted |
| 56 | Ulcerative colitis | Suspected |
| 57 | Urticaria | Accepted |
| 58 | Uveitis | Accepted |
| 59 | Vasculitis | Accepted |
| 60 | Vitiligo | Accepted |
| 61 | Wegener's granulomatosis | Accepted |

### Aetiology of ADs

Although the aetiology is unclear all ADs are caused by failure of the mechanisms that regulate immune system function, which results in autoimmune attack on organs and tissue in the body. The diseases also share certain similarities at the molecular level (Karopka, T. et al., BMC Bioinformatics. 7: (2006); van der Pouw Kraan, T. C. T. M. et al., Ann. Rheum. Dis. 66: 1008-1014 (2007)). Patients suffering from one AD often suffer from one or more ADs (Tsuneyama, K. et al., Dig. Dis. Sci. 45: 366-372 (2000)). An elevated risk of four separate autoimmune diseases, T1D, RA, Systemic lupus erythematosus (SLE) and autoimmune thyroid disease (AITD) (Hashimotos Thyroiditis or Graves disease), were found in families with multiple autoimmune diseases (Criswell, L. A. et al., Am. J. Hum. Genet. 76: 561-571 (2005)). Studies on rheumatoid arthritis (RA) and schizophrenia suggest they share a common infectious and/or immune aetiology (Torrey, E. F. et al., Brain Behav. Immun. 15: 401-410 (2001); Eaton, W. W. et al., Am. J. Psychiat. 163: 521-528 (2006); Eaton, W. W. et al., J. Autoimmun. 29: 1-9 (2007)).

Relevant animal models are crucial for studying the aetiology, pathogenesis and treatment of ADs (Burkhardt, H. et al., Rheumatol. Int. 17: 85-90 (1997)), although there are difficulties in choosing an appropriate animal model due to aetiological uncertainties (Drescher, K. M. et al., Front. Biosci. 13: 3775-3785 (2008)).

The diabetes-prone BB rat develops T1D in a manner that is similar to human T1D (Jacob, H. J. et al., Nature genetics. 2: 56-60 (1992); Mordes, J. P. et al., Ilar Journal. 45: 278-291 (2004); Fuller, J. M. et al., Diabetes. 55: 3351-3357 (2006)). Rodent models are also important tools in research into the pathogenesis of RA (Terato, K. et al., Br. J. Rheumatol. 35: 828-838 (1996); Burkhardt, H. et al., Rheumatol. Int. 17: 85-90 (1997); Anthony, D. D. et al., Clin. Exp. Rheumatol. 17: 240-244 (1999); Holmdahl, R. et al., Immunol. Rev. 184: 184-202 (2001); Nandakumar, K. S. et al., Arthritis Res. Ther. 8: (2006)) and have also been the base for research on susceptibility genes (Remmers, E. F. et al., Nature genetics. 14: 82-85 (1996); Holmdahl, R., Curr. Opin. Immunol. 10: 710-717 (1998)). Larger animal models with organ systems resembling those of humans are an important complement in both diabetes and arthritis research (Larsen, M. O. et al., Ear Journal. 45: 303-313 (2004); Vierboom, M. P. M. et al., Arthritis Res. Ther. 7: 145-154 (2005)).

### Groups and Subgroups

The common feature of all ADs is systemic failure resulting in autoimmune attack on cells and tissue, which can affect any organ in the body.

The following accepted or suspected ADs have been linked in scientific studies and have common denominators relating to aetiology or the affected organs: 1, 2, 4, 9, 10, 12, 16, 20, 21, 22, 23, 25, 27, 28, 29, 35, 37, 38, 39, 45, 46, 48, 49, 52, 53, 55 and 58. More specifically the ADs of this subgroup have reported links to arthritic conditions and are generally characterised by degradation of e.g. myelin or connective tissue in e.g. joints and skin.

Furthermore, a subgroup of the following accepted or suspected ADs have been linked in scientific studies and have common denominators relating to aetiology or the affected organs: 4, 9, 16, 20, 35, 37, 39, 45, 46, 48, 49, 52, 53 and 58. The ADs of this subgroup have reported links to arthritic conditions that are characterised by degradation of connective tissue, especially in the joints, skin and eyes.

Furthermore, a subgroup of the following accepted or suspected ADs have been linked in scientific studies and have common denominators relating to aetiology or the affected organs: 1, 12, 28, 37 and 38. The ADs of this subgroup are all affected by degradation of myelin of the central nervous system.

Furthermore, a subgroup of the following accepted or suspected ADs have been linked in scientific studies and have common denominators relating to aetiology or the affected organs: 13, 19, 20, 24, 32, 36, 40, 45, 46, 51, 57 and 60. The ADs of this subgroup all affect the skin by degradation of e.g. connective tissue or hardening and hardening of the skin. The psoriatic conditions are linked to the arthritic conditions.

Furthermore, a subgroup of the following accepted or suspected ADs have been linked in scientific studies and have common denominators relating to aetiology or the affected organs: 14, 18, 41 and 56. The ADs of this subgroup all affect the digestive system by e.g. degradation of bowel tissue. The conditions in this subgroup have been linked to Lupus, an arthritic condition.

Furthermore, a subgroup of the following accepted or suspected ADs have been linked in scientific studies and have common denominators relating to aetiology or the affected organs: 5 6 11 30 31 33 42, 54 and 59. The ADs of this subgroup all affect the cardiovascular system by e.g. degradation of blood vessels. The conditions in this subgroup have been linked to arthritic conditions.

Furthermore, a subgroup of the following accepted or suspected ADs have been linked in scientific studies and have common denominators relating to aetiology or the affected organs: 1, 7, 47 and 50. The ADs of this subgroup all affect the neurological system by e.g. causing inflammatory lesions in the brain. The conditions in this subgroup have been linked to arthritic conditions and MS.

Furthermore, a subgroup of the following accepted or suspected ADs have been linked in scientific studies and have common denominators relating to aetiology or the affected organs: 21, 25, 26, 34 and 61. The ADs of this subgroup all affect the kidneys and/or lungs by e.g. causing inflammatory destruction of blood vessels in these organs. The conditions in this subgroup have been linked to arthritic conditions, T1D and MS.

In one embodiment, the invention relates to a method as described herein for the detection of an elevated risk for, diagnosis or prognosis of any of the above subgroups of diseases.

### Diagnosis of AD

ADs can affect any organ or tissue in the body and in most cases symptoms are not perceptible until the disease is in an advanced stage and irreversible damage has occurred. To date AD is almost exclusively a clinical diagnosis, which is made difficult by varying and unspecific early symptoms (Health, National Institute of: (2002)). For most ADs the biomarkers, if they exist, are not specific and hence there is limited availability of laboratory based tests to aid in diagnosis, which also hampers clinical management and development of new therapeutic agents (Liu, C. C. et al., Curr. Opin. Rheumatol. 17: 543-549 (2005)).

Autoantibodies have been identified as clinically relevant biomarkers for SLE and other ADs (Ramos, P. S. et al., Genes Immun. 7: 417-432 (2006)).

Cytokines have been associated with ADs and have been used to discriminate ADEM (acute disseminated encephalomyelitis) (Wingerchuk, D. M., Neurol. Res. 28: 341-347 (2006)), MS (multiple sclerosis) and healthy controls (Franciotta, D. et al., J. Neurol. Sci. 247: 202-207 (2006); Wingerchuk, D. M. et al., Curr. Opin. Neurol. 20: 343-350 (2007)). Cytokines are also involved in the pathogenesis of RA (Ehrenstein, M. R. et al., J. Exp. Med. 200: 277-285 (2004); McInnes, I. B. et al., Nat. Rev. Immunol. 7: 429-442 (2007)).

### Treatment of AD

Currently, there are no cures available for most ADs, and patients can expect a lifetime of disease and treatment to reduce the symptoms. Two main approaches to treatment are available:
replacing the damaged organ or repairing impaired functions
suppressing the destructive autoimmune response

In the first category one example is insulin for T1D patients, which will not cure the AD. To restore pancreas islet cell mass T1D patients may undergo surgery to have pancreas or islet transplantation (Sutherland, D. E. R. et al., Diabetes. 38: 85-87 (1989); Kandaswamy, R. et al., Transplant. Proc. 38: 365-367 (2006); Sutherland, D. E. R. et al., Transplant. Proc. 39: 2323-2325 (2007)) with novel approaches including regeneration from stem cells and pancreatic progenitor cells (Jun, H. S., Front. Biosci. 13: 6170-6182 (2008)).

The second category deals with improving signs and symptoms, e.g. with non-steroidal anti-inflammatory drugs and simple analgesics, modifying the natural course of disease, e.g. disease modifying anti-rheumatic drugs (DMARDs) for RA patients, and addressing complications resulting from organ damage brought about by the disease. The development of biomarkers for ADs is of critical importance to determine the stage, activity and progression of disease and to assess response to therapy.

### Type 1 Diabetes Mellitus

T1D is one of the most common chronic diseases among young children with decreasing age of onset and an estimated global increase of 3-5% per year; the need for early and better diagnostic tools is obvious (Onkamo, P. et al., Diabetologia. 42: 1395-1403 (1999); Gale, E. A. M., Diabetes. 51: 3353-3361 (2002); Gillespie, K. M. et al., Lancet. 364: 1699-1700 (2004)). T1D is a T-cell mediated AD that begins, in many cases, three to five years before the onset of clinical symptoms, continues after diagnosis, and can recur after islet transplantation (Eisenbarth, G. S., N. Engl. J. Med. 314: 1360-1368 (1986); Tyden, G. et al., N. Engl. J. Med. 335: 860-863 (1996); Atkinson, M. A. et al., Lancet. 358: 221-229 (2001)). The aetiology is not known, but theories link genetic and environmental factors and possibly virus infections to T1D (Kyvik, K. O. et al., Br. Med. J. 311: 913-917 (1995); Åkerblom, H. K. et al., Am. J. Med. Genet. 115: 18-29 (2002); Barbeau, W. E. et al., Med. Hypotheses. 68: 607-619 (2007)).

T1D is viewed as a two-step process (FIG. 1) involving triggering of an autoimmune process (step one) resulting in islet autoimmunity, followed by progression to clinical onset of hyperglycemia (step two) (Dahlquist, G. G. et al., Diabetes. 44: 408-413 (1995); Hyöty, H. et al., Diabetes. 44: 652-657 (1995); Lindberg, B. et al., Diabetologia. 42: 181-187 (1999); Skyler, J. S. et al., N. Engl. J. Med. 346: 1685-1691B (2002); Gale, E. A. M. et al., Lancet. 363: 925-931 (2004)).

The majority of patients are not diagnosed until they reach the endpoint of a prolonged autoimmune and early diagnosis would lead to better-controlled blood glucose levels and preservation of the remaining endogenous insulin production and would also reduce the risk of future complications (Livingstone, K. et al., Practical Diabetes Int. 24: 102-106 (2007)). In order to identify risk factors newborns have been screened for HLA (human leukocyte antigen) in several studies to identify and follow those children with T1D-high risk HLA (Krischer, J., Pediatr. Diabetes. 8: 286-298 (2007)). Appearance of islet autoantibodies and their prediction of diabetes among children with a first degree relative with T1D have been reported (Achenbach, P. et al., Diabetes. 53: 384-392 (2004); Barker, J. M. et al., J. Clin. Endocrinol. Metab. 89: 3896-3902 (2004); Ronkainen, M. S. et al., Eur. J. Endocrinol. 155: 633-642 (2006)), although approximately 85% of all children developing T1D do not have a first degree relative with the disease. Apart from the appearance of islet autoimmunity there are observations indicating that metabolic changes including growth may be associated with the T1D disease process (Shaham, Oded et al., Mol Syst Biol. 4: (2008)).

TABLE 2
From WO 2008/035204

| Metabolite | Significance, p |
|---|---|
| Total carnitine | 0.004 |
| Free carnitine | 0.009 |
| Acylcarnitine | 0.009 |
| Acyl/free ratio | 0.556 |
| Alanine (Ala) | 0.037 |
| Arginine (Arg) | 0.599 |
| Aspartate (Asp) | 0.461 |
| Citrulline (Cit) | 0.064 |
| Glycine (Gly) | 0.002 |
| Glutamate/Glutamine (Glu/Gln) | 0.002 |
| *Leucine/Isoleucine (Leu/Xle) | <0.001 |
| *Methionine (Met) | 0.326 |
| Ornithine (Orn) | 0.001 |
| *Phenylalanine (Phe) | 0.001 |
| Proline (Pro) | 0.002 |
| Thyrosine (Thy) | 0.323 |
| *Valine (Val) | 0.192 |
| Essential amino acids | 0.003 |
| Non essential amino acids | 0.003 |
| Total amino acids | 0.003 |

*Essential amino acids (excluding arginine)

Prediction of T1D and biomarkers for T1D risk has been reported in a number of inventions (e.g. WO 2008/031917, US2005054005, WO2006066263, WO2007110358 and DE102006026173). One invention claiming prediction of T1D (WO 2008/035204) in subjects involves different metabolites and the additional step of genotyping, see Table 2. To our surprise we found that samples from subjects with an elevated risk of developing T1D contain novel metabolites and unique metabolite combinations that have not previously been associated with risk of T1D onset, and that the observed variation in the levels of these metabolites over time is a hallmark of elevated risk of T1D.

In diabetes mellitus type 2 (T2D) the cells of the body develop an insulin resistance and hence do not respond appropriately to the presence of insulin. However, there are a number of similarities between T1D and T2D that links the aetiology and pathogenesis of the diseases (Tuomi, T. et al., Diabetes. 42: 359-362 (1993); Pickup, J. C. et al., Diabetologia. 41: 1241-1248 (1998); Xing, Z. et al., J. Clin. Invest. 101: 311-320 (1998); Rabinovitch, A. et al., Rev. Endocr. Metab. Disord. 4: 291-299 (2003); Alexandraki, K. I. et al., J. Clin. Immunol. 28: 314-321 (2008))

### Rheumatoid Arthritis

RA is a chronic, inflammatory AD that causes the immune system to attack the joints, which results in a disabling and painful condition that can lead to substantial loss of mobility due to joint destruction and the associated pain (Scott, D. L. et al., Best Pract. Res. Clin. Rheumatol. 21: 943-967 (2007)). The aetiology behind RA is largely unknown (Jefferies, W. M., Medical Hypotheses. 51: 111-114 (1998); Krishnan, E., Joint Bone Spine. 70: 496-502 (2003); Klareskog, L. et al., Arthritis and Rheumatism. 54: 38-46 (2006)). It is likely that the first symptoms are undifferentiated inflammatory arthritis (UIA) rather than RA, and that the UIA may evolve into RA (Dixon, W. G. et al., Best Pract. Res. Clin. Rheumatol. 19: 37-53 (2005)). The evolution of inflammatory arthritis can be divided into four stages (FIG. 2).

Early diagnosis is crucial for more effective treatment to prevent irreversible joint damage (Emery, P., Br. J. Rheumatol. 33: 765-768 (1994); van Aken, J. et al., Ann. Rheum. Dis. 63: 274-279 (2004); Emery, P., Br. Med. J. 332: 152-155 (2006)). Detection of patients who go on to develop chronic joint inflammation would aid in targeting those in need of treatment and avoid unnecessary treatment of those less likely to develop the disease (Emery, P. et al., Rheumatol. Int. 27: 793-806 (2007); Emery, P. et al., Rheumatology. 47: 392-398 (2008)). Hence there is a need for methods to diagnose the autoimmune response which is UIA before it evolves into RA (Firestein, G. S., Arthritis Res. Ther. 7: 157-159 (2005)). Measuring concentrations of endogenous metabolites has the potential to not only diagnose the disease but also to provide new clues to the mechanisms involved in pathogenesis. In the examples we show that it is possible to separate subjects suffering from RA from subjects with related diseases and healthy volunteers by analyzing data generated by GC-MS (gas chromatography-mass spectrometry) of blood plasma, see Table 3. This is crucial for development of drugs that act to delay or even prevent the onset of RA.

TABLE 3
Metabolites from human RA

| Metabolite | Direction in RA | Significance, p |
|---|---|---|
| Isoleucine | ↓ | <0.001 |
| Glycine | ↓ | 0.03 |
| Succinate | ↓ | <0.001 |
| Glyceric acid | ↓ | <0.001 |
| Serine | ↓ | <0.001 |
| Threonine | ↓ | <0.001 |
| Malic acid | ↓ | <0.001 |
| Methionine | ↓ | 0.002 |
| Aspartate | ↓ | <0.001 |
| Pyroglutamate | ↓ | <0.001 |
| 4-Hydroxyproline | ↓ | <0.001 |
| Threonic acid | ↓ | 0.005 |
| Glutamate | ↓ | <0.001 |
| Phenylalanine | ↑ | 0.62 |
| Aspargine | ↓ | <0.001 |
| Glutamine | ↑ | 0.64 |
| Ornithine | ↓ | <0.001 |
| Glucose | ↑ | <0.001 |
| Lysine | ↓ | <0.001 |
| Tyrosine | ↓ | 0.001 |
| Tryptophan | ↓ | <0.001 |
| alfa-tocopherol | ↑ | <0.001 |

### Osteoarthritis

Osteoarthritis (OA) is the most common musculoskeletal disorder world-wide and it is estimated that nearly 27 million are affected in the US alone (Ghosh, P. et al., Biogerontology. 3: 85-88 (2002); Lawrence, R. C. et al., Arthritis and Rheumatism. 58: 26-35 (2008)). OA is the major cause of morbidity in the developed world with massive social and economic consequences (Stargardt, T., Health Econ. 17: S9-S20 (2008)).

OA is a multifactorial disease characterized by progressive degeneration and loss of articular cartilage and subchondral bone, and synovial reaction. The pathogenesis and variability of OA are poorly understood, but it is believed that age, genetic, hormonal and mechanical factors all contribute to the onset and progression.

A number of studies have found evidence of autoimmunity in OA (Nishioka, K., Arthritis Res. Ther. 6: 6-7 (2004); Punzi, L. et al., Best Pract. Res. Clin. Rheumatol. 18: 739-758 (2004); Xiang, Y. et al., Arthritis Rheum. 50: 1511-1521 (2004); Xiang, Y. et al., J. Immunol. 176: 3196-3204 (2006)), especially for the sub-group of nodal generalised AO (Doherty, M. et al., Ann Rheum Dis. 49: 1017-1020 (1990)).

The diagnosis of OA almost always involves radiographic assessment of joint damage, which is the result of several months of disease progression. Radiographic evidence occurs in the majority of people by 65 years of age and in about 80% of those aged over 75 years (Arden, N. et al., Best Pract. Res. Clin. Rheumatol. 20: 3-25 (2006)). This method of diagnosis is not suited for assessment of current disease activity or for prognosis and hence there is a need for biomarkers that enable early diagnosis of OA and the autoimmune response which is associated with OA. This is necessary to allow for an assessment of risk of onset, monitoring of disease progression and severity, especially in connection with development of effective agents to treat OA. Measuring concentrations of endogenous metabolites have the potential to not only diagnose the disease but also to provide new clues to the mechanisms involved in pathogenesis. In the examples we show that it is possible to separate OA-patients from healthy individuals, see Table 4. This may aid in the development of treatment to delay or even prevent the onset of OA.

TABLE 4
Metabolites from human OA in Example 2

| Metabolite | Direction in OA | Significance, p |
|---|---|---|
| Phosphoric acid | ↑ | <0.001 |
| Isoleucine | ↓ | <0.001 |
| Glycine | ↓ | 0.002 |
| Succinate | ↓ | <0.001 |
| Serine | ↓ | <0.001 |
| Threonine | ↓ | 0.002 |
| Malic acid | ↓ | <0.001 |
| Methionine | ↓ | 0.002 |
| Aspartate | ↓ | <0.001 |
| Pyroglutamate | ↓ | <0.001 |
| 4-Hydroxyproline | ↓ | <0.001 |
| Glutamate | ↓ | <0.001 |
| Phenylalanine | ↓ | <0.001 |
| Aspargine | ↓ | <0.001 |
| Glycerol-3-phosphate | ↓ | <0.001 |
| Ornithine | ↓ | <0.001 |
| Glucose | ↑ | <0.001 |
| Lysine | ↓ | <0.001 |
| Tyrosine | ↓ | 0.001 |
| Tryptophan | ↓ | 0.002 |
| Inositol-1-phosphate | ↑ | 0.002 |
| alfa-tocopherol | ↑ | <0.001 |
| Sterol | ↓ | <0.001 |

A set of endogenous metabolites has now been found that identifies subjects at risk of developing an AD. A subject's profile of these endogenous metabolites may therefore aid in the prognosis, detection and diagnosis of AD, which can be carried out before any clinical symptoms occur, and this enables preventative treatment or prophylaxis to be started early so as to minimise any longer-term tissue damage. The profile of endogenous metabolites may also be used as a tool for screening and identification of drugs and new chemical entities (NCEs) which can act to restore normal levels of these endogenous metabolites, thereby preventing or delaying the onset of the AD and thus being efficacious in the treatment of AD.

We have identified a compound which in our tests has shown ability restore endogenous metabolite levels and which will therefore be of benefit in preventing, delaying or reducing the onset of diseases or disorders, particularly those believed of autoimmune origin, in many of its forms.

The terms “biomarker” and “endogenous metabolite”, and the plurals thereof, are used herein interchangeably.

In one embodiment, the invention provides a method for the detection of a disturbance in the metabolic profile of endogenous metabolites in a subject caused by rheumatoid arthritis (RA), the method comprising:

(i) measuring the levels of N endogenous metabolites in a biological sample obtained from the subject, wherein N is 2-11, in order to produce a metabolic profile of the N endogenous metabolites in that subject;
(ii) comparing the measured levels of the N endogenous metabolites with the corresponding levels of the endogenous metabolites in a biological sample obtained from a control;
wherein the N endogenous metabolites are selected from the group consisting of:

| | |
|---|---|
| EM1 | Phenylalanine |
| EM2 | Tyrosine |
| EM3 | Isoleucine |
| EM8 | Glycine |
| EM9 | Glutamine |
| EM10 | Methionine |
| EM14 | Lysine |
| EM15 | Asparagine |
| EM16 | Serine |
| EM17 | Tryptophan |
| EM18 | Threonine |

wherein the disturbance in the metabolic profile is one wherein there is independently a decrease or increase in the level of each of the N measured endogenous metabolites in the biological sample obtained from the subject compared to the corresponding levels of endogenous metabolites in the biological sample obtained from the control, and wherein the disturbance in the metabolic profile is due to RA in the subject, and optionally, if a disturbance in the metabolic profile is detected,
(iiia) prescribing or supplying to the subject or recommending treatment of the subject with an effective amount of N-(2-chloro-3,4-dimethoxybenzylideneamino)guanidine or an aminoguanidine, either as the free base or in salt form, for normalising the disturbed metabolic profile; or
(iiib) administering to said subject an effective amount of N-(2-chloro-3,4-dimethoxybenzylideneamino)guanidine or an aminoguanidine, either as the free base or in salt form, for normalising the disturbed metabolic profile.

The invention also provides a method for the detection of a disturbance in the metabolic profile of endogenous metabolites in a subject caused by rheumatoid arthritis (RA), the method comprising:

(i) measuring the levels of N endogenous metabolites in a biological sample obtained from the subject, wherein N is 2-11, in order to produce a metabolic profile of the N endogenous metabolites in that subject;
(ii) comparing the measured levels of the N endogenous metabolites with the corresponding levels of the endogenous metabolites in a biological sample previously obtained from the subject;
wherein the N endogenous metabolites are selected from the group consisting of:

| | |
|---|---|
| EM1 | Phenylalanine |
| EM2 | Tyrosine |
| EM3 | Isoleucine |
| EM8 | Glycine |
| EM9 | Glutamine |
| EM10 | Methionine |
| EM14 | Lysine |
| EM15 | Asparagine |
| EM16 | Serine |
| EM17 | Tryptophan |
| EM18 | Threonine |

wherein the disturbance in the metabolic profile is one wherein there is a independently a decrease or increase in the level of each of the N measured endogenous metabolites in the biological sample obtained from the subject compared to the corresponding levels of endogenous metabolites in the biological sample previously obtained from the subject; and wherein the disturbance in the metabolic profile is due to RA in the subject, and optionally, if a disturbance in the metabolic profile is detected,
(iiia) prescribing or supplying to the subject or recommending treatment of the subject with an effective amount of N-(2-chloro-3,4-dimethoxybenzylideneamino)guanidine or an aminoguanidine, either as the free base or in salt form, for normalising the disturbed metabolic profile; or
(iiib) administering to said subject an effective amount of N-(2-chloro-3,4-dimethoxybenzylideneamino)guanidine or an aminoguanidine, either as the free base or in salt form, for normalising the disturbed metabolic profile.

In a further embodiment, the invention provides a method of diagnosing or detecting RA in a subject, the method comprising:

(i) measuring the levels of N biomarkers in a biological sample obtained from the subject, wherein N is 2-11;
(ii) comparing the measured levels of the N biomarkers with the corresponding levels of the biomarkers in a biological sample obtained from a control;
wherein the N biomarkers are selected from the group consisting of:

| | |
|---|---|
| EM1 | Phenylalanine |
| EM2 | Tyrosine |
| EM3 | Isoleucine |
| EM8 | Glycine |
| EM9 | Glutamine |
| EM10 | Methionine |
| EM14 | Lysine |
| EM15 | Asparagine |
| EM16 | Serine |
| EM17 | Tryptophan |
| EM18 | Threonine |

wherein a decrease in the level of each of the N measured biomarkers in the biological sample obtained from the subject compared to the corresponding levels of biomarkers in the biological sample obtained from the control is indicative of the presence of RA in the subject.

A further aspect of the invention provides a method of monitoring RA progression in a subject, the method comprising:

(i) measuring the levels of N biomarkers in a biological sample obtained from the subject, wherein N is 2-11;
(ii) comparing the measured levels of the N biomarkers with the corresponding levels of the biomarkers in a biological sample previously obtained from the subject;
wherein the N biomarkers are selected from the group consisting of:

| | |
|---|---|
| EM1 | Phenylalanine |
| EM2 | Tyrosine |
| EM3 | Isoleucine |
| EM8 | Glycine |
| EM9 | Glutamine |
| EM10 | Methionine |
| EM14 | Lysine |
| EM15 | Asparagine |
| EM16 | Serine |
| EM17 | Tryptophan |
| EM18 | Threonine |

wherein differences in the levels of the N measured biomarkers in the biological sample obtained from the subject compared to the corresponding levels of biomarkers in the biological sample previously obtained from the subject is indicative of a change in the RA prognosis in the subject.

In particular, an increase in the level of each of the N measured biomarkers is indicative of an improvement in the RA prognosis in the subject.

Furthermore, a decrease in the level of each of the N measured biomarkers is indicative of a decline in the RA prognosis in the subject.

The invention also provides a method for monitoring the rate of decline or rate of improvement of RA in a subject, comprising a method for monitoring RA progression as described herein, comprising the additional step of dividing the increase or decrease of biomarker levels by the time interval between the taking of the first (i.e. previous) and second samples from the subject.

A further aspect of the invention provides a method of measuring the effectiveness of a medicament which has been administered to a subject to treat RA in that subject, the method comprising:

(i) measuring the levels of N biomarkers in a biological sample obtained from the treated subject, wherein N is 2-11;
(ii) comparing the measured levels of the N biomarkers with the corresponding levels of the biomarkers in a biological sample previously obtained from the subject;
wherein the N biomarkers are selected from the group consisting of:

| | |
|---|---|
| EM1 | Phenylalanine |
| EM2 | Tyrosine |
| EM3 | Isoleucine |
| EM8 | Glycine |
| EM9 | Glutamine |
| EM10 | Methionine |
| EM14 | Lysine |
| EM15 | Asparagine |
| EM16 | Serine |
| EM17 | Tryptophan |
| EM18 | Threonine |

wherein differences in the levels of the N measured biomarkers in the biological sample obtained from the treated subject compared to the corresponding levels of biomarkers in the biological sample previously obtained from the subject provide an indication of the efficacy of the medicament in that subject.

In particular, an increase in the level of each of the N measured biomarkers is indicative of the medicament being efficacious.

Furthermore, a decrease in the level of each of the N measured biomarkers is indicative of a lack of efficacy of the medicament.

A further aspect of the invention provides a method of measuring the effectiveness of a medicament which has been administered to a subject to treat RA in that subject, the method comprising:

(i) measuring the levels of N biomarkers in a biological sample obtained from the treated subject, wherein N is 2-11;
(ii) comparing the measured levels of the N biomarkers with the corresponding levels of the biomarkers in a biological sample obtained from a control;
wherein the N biomarkers are selected from the group consisting of:

| | |
|---|---|
| EM1 | Phenylalanine |
| EM2 | Tyrosine |
| EM3 | Isoleucine |
| EM8 | Glycine |
| EM9 | Glutamine |
| EM10 | Methionine |
| EM14 | Lysine |
| EM15 | Asparagine |
| EM16 | Serine |
| EM17 | Tryptophan |
| EM18 | Threonine |

wherein differences in the levels of the N measured biomarkers in the biological sample obtained from the treated subject compared to the corresponding levels of biomarkers in the biological sample obtained from the control provide an indication of the efficacy of the medicament in that subject.

In particular, an increase in the level of each of the N measured biomarkers is indicative of the medicament being efficacious.

Furthermore, a decrease in the level of each of the N measured biomarkers is indicative of a lack of efficacy of the medicament.

The invention also provides a method for monitoring the effectiveness of a medicament which has been administered to a subject to treat RA as described above, which comprises the additional step of administering the medicament to the subject prior to step (i) or in the interval between the taking of samples.

In the context of the above methods for monitoring the effectiveness of a medicament, in some embodiments of the invention, the medicament has been administered to the subject before the two biological samples have been obtained. In other embodiments, the medicament has been administered to the subject in the interval between the taking of the two samples.

In some embodiments of the invention, the term “method of diagnosing or detecting RA” means a method for detecting probable RA in a subject or a method of determining an increased likelihood of RA in a subject.

Preferably N is 3-11, 4-11, 5-11, 6-11, 7-11, 8-11, 9-11 or 10-11. In other embodiments, N is 2-5, 3-5 or 4-5. In yet other embodiments, N is 2, 3, 4, 5, 6, 7, 8, 9, 10 or 11. In some embodiments, the endogenous metabolites are selected from EMs 1-3 and/or EMs 8-10 and/or EMs 14-18; or the endogenous metabolites are selected from EM2, EM3, EM8 and EM10.

For example, if the embodiment of the invention requires N endogenous metabolites to be selected, the endogenous metabolites may be selected from EMs 1-N.

An increased level of confidence in the method can be obtained using methods where N is a higher value.

In a further embodiment, the invention provides a method for the detection of a disturbance in the metabolic profile of endogenous metabolites in a subject caused by an autoimmune disease, the method comprising:

(i) measuring the levels of N endogenous metabolites in a biological sample obtained from the subject, wherein N is 2-13, in order to produce a metabolic profile of the N endogenous metabolites in that subject;
(ii) comparing the measured levels of the N endogenous metabolites with the corresponding levels of the endogenous metabolites in a biological sample obtained from a control;
wherein the N endogenous metabolites are selected from the group consisting of:

| | |
|---|---|
| EM1 | Phenylalanine (Phe) |
| EM2 | Tyrosine (Tyr) |
| EM3 | Isoleucine (Ile) |
| EM4 | Leucine (Leu) |
| EM5 | Ornithine (Orn) |
| EM6 | Proline (Pro) |
| EM7 | Glutamate (Glu) |
| EM8 | Glycine (Gly) |
| EM9 | Glutamine (Gln) |
| EM10 | Methionine (Met) |
| EM11 | Aspartate (Asp) |
| EM12 | Valine (Val) |
| EM13 | Arginine (Arg) |

wherein the disturbance in the metabolic profile is one wherein there is a independently a decrease or increase in the level of each of the N measured endogenous metabolites in the biological sample obtained from the subject compared to the corresponding levels of endogenous metabolites in the biological sample obtained from the control;
wherein the disturbance in the metabolic profile is due to:
(a) an autoimmune disease, preferably all autoimmune diseases;
(b) all autoimmune diseases or disorders which are characterised by tissue degradation;
(c) all autoimmune diseases or disorders which are characterised by degradation of connective tissue;
(d) all autoimmune diseases or disorders which are characterised by degradation of myelin in the central nervous system;
(e) all autoimmune diseases or disorders which affect the skin by degradation;
(f) all autoimmune diseases or disorders which affect the digestive system;
(g) all autoimmune diseases or disorders which affect the cardiovascular system;
(h) all autoimmune diseases or disorders which affect the neurological system;
(i) all autoimmune diseases or disorders which affect the kidneys and/or lungs;
(j) all diabetic disorders; or
(k) all arthritic disorders,
and optionally, if a disturbance in the metabolic profile is detected,
(iiia) prescribing or supplying to the subject or recommending treatment of the subject with an effective amount of N-(2-chloro-3,4-dimethoxybenzylideneamino)guanidine or an aminoguanidine, either as the free base or in salt form, for normalising the disturbed metabolic profile; or
(iiib) administering to said subject an effective amount of N-(2-chloro-3,4-dimethoxybenzylideneamino)guanidine or an aminoguanidine, either as the free base or in salt form, for normalising the disturbed metabolic profile.

In a further embodiment, the invention provides a method for the detection of a disturbance in the metabolic profile of endogenous metabolites in a subject caused by an autoimmune disease, the method comprising:

(i) measuring the levels of N endogenous metabolites in a biological sample obtained from the subject, wherein N is 2-13, in order to produce a metabolic profile of the N endogenous metabolites in that subject;
(ii) comparing the measured levels of the N endogenous metabolites with the corresponding levels of the endogenous metabolites in a biological sample previously obtained from the subject;
wherein the N endogenous metabolites are selected from the group consisting of:

| | |
|---|---|
| EM1 | Phenylalanine (Phe) |
| EM2 | Tyrosine (Tyr) |
| EM3 | Isoleucine (Ile) |
| EM4 | Leucine (Leu) |
| EM5 | Ornithine (Orn) |
| EM6 | Proline (Pro) |
| EM7 | Glutamate (Glu) |
| EM8 | Glycine (Gly) |
| EM9 | Glutamine (Gln) |
| EM10 | Methionine (Met) |
| EM11 | Aspartate (Asp) |
| EM12 | Valine (Val) |
| EM13 | Arginine (Arg) |

wherein the disturbance in the metabolic profile is one wherein there is independently a decrease or increase in the level of each of the N measured endogenous metabolites in the biological sample obtained from the subject compared to the corresponding levels of endogenous metabolites in the biological sample previously obtained from the subject;
and wherein the disturbance in the metabolic profile is due to one or more of the diseases or disorders defined above,
and optionally, if a disturbance in the metabolic profile is detected,
(iiia) prescribing or supplying to the subject or recommending treatment of the subject with an effective amount of N-(2-chloro-3,4-dimethoxybenzylideneamino)guanidine or an aminoguanidine, either as the free base or in salt form, for normalising the disturbed metabolic profile; or
(iiib) administering to said subject an effective amount of N-(2-chloro-3,4-dimethoxybenzylideneamino)guanidine or an aminoguanidine, either as the free base or in salt form, for normalising the disturbed metabolic profile.

Preferably, the disturbance in the metabolic profile is due to a diabetic disorder, most preferably Type 2 diabetes (T2D).

In other preferred embodiments, the disturbance is due to an autoimmune disease which is characterised by inflammation, particularly inflammation of the joints.

In yet another embodiment, the invention provides a method for the detection of an elevated risk for, diagnosis or prognosis of a disease or disorder in a subject, the method comprising:

(i) measuring the levels of N biomarkers in a biological sample obtained from the subject, wherein N is 2-13;
(ii) comparing the measured levels of the N biomarkers with the corresponding levels of the biomarkers in a biological sample obtained from a control;
wherein the N biomarkers are selected from the group consisting of:

| | |
|---|---|
| EM1 | Phenylalanine (Phe) |
| EM2 | Tyrosine (Tyr) |
| EM3 | Isoleucine (Ile) |
| EM4 | Leucine (Leu) |
| EM5 | Ornithine (Orn) |
| EM6 | Proline (Pro) |
| EM7 | Glutamate (Glu) |
| EM8 | Glycine (Gly) |
| EM9 | Glutamine (Gln) |
| EM10 | Methionine (Met) |
| EM11 | Aspartate (Asp) |
| EM12 | Valine (Val) |
| EM13 | Arginine (Arg) |

wherein a decrease in the level of each of the N measured biomarkers in the biological sample obtained from the subject compared to the corresponding levels of biomarkers in the biological sample obtained from the control provides an indication of an elevated risk for, a diagnosis of or reduced prognosis of a disease or disorder in the subject.

In a further embodiment, the invention provides a method for detection of an elevated risk for, diagnosis or prognosis of a disease or disorder in a subject, the method comprising:

(i) measuring the levels of N biomarkers in a biological sample obtained from the subject, wherein N is 2-13;
(ii) comparing the measured levels of the N biomarkers with the corresponding levels of the biomarkers in a biological sample previously obtained from the subject;
wherein the N biomarkers are selected from the group consisting of:

| | |
|---|---|
| EM1 | Phenylalanine (Phe) |
| EM2 | Tyrosine (Tyr) |
| EM3 | Isoleucine (Ile) |
| EM4 | Leucine (Leu) |
| EM5 | Ornithine (Orn) |
| EM6 | Proline (Pro) |
| EM7 | Glutamate (Glu) |
| EM8 | Glycine (Gly) |
| EM9 | Glutamine (Gln) |
| EM10 | Methionine (Met) |
| EM11 | Aspartate (Asp) |
| EM12 | Valine (Val) |
| EM13 | Arginine (Arg) |

wherein a decrease in the level of each of the N measured biomarkers in the biological sample obtained from the subject compared to the corresponding levels of biomarkers in the biological sample previously obtained from the subject is indicative of an elevated risk for, a diagnosis of or reduced prognosis of the disease or disorder in the subject.

As used herein, the term “disease or disorder” refers to:

(a) all autoimmune diseases;
(b) all autoimmune diseases or disorders which are characterised by tissue degradation, for example myelin or connective tissue in e.g. joints and skin;
(c) all autoimmune diseases or disorders which are characterised by degradation of connective tissue, for example in the joints, skin and eyes;
(d) all autoimmune diseases or disorders which are characterised by degradation of myelin in the central nervous system;
(e) all autoimmune diseases or disorders which affect the skin by degradation, for example of connective tissue or hardening of the skin;
(f) all autoimmune diseases or disorders which affect the digestive system, for example by degradation of bowel tissue;
(g) all autoimmune diseases or disorders which affect the cardiovascular system, for example by degradation of blood vessels;
(h) all autoimmune diseases or disorders which affect the neurological system, for example by causing inflammatory lesions in the brain;
(i) all autoimmune diseases or disorders which affect the kidneys and/or lungs, for example by causing inflammatory destruction of blood vessels in these organs;
(j) all diabetic disorders, for example T1D and T2D;
(k) all arthritic disorders, for example OA and RA; or

### (l) T1D, RA and OA.

In some embodiments of the invention, EM6 proline (Pro) is replaced by 4-hydroxyproline. In other embodiments, EM6 is the combined level of proline and 4-hydroxyproline in the relevant sample.

Examples of the diseases or disorders referred to in (b)-(i) may be found above in the section headed “Groups and subgroups” and in the corresponding diseases mentioned in Table 1.

In some embodiments, a decrease in the level of each of the N measured biomarkers is indicative of the diagnosis of the disease or disorder in the subject.

In some embodiments, a decrease in the level of each of the N measured biomarkers is indicative of a decline in the prognosis of the disease or disorder in the subject.

In some embodiments, an increase in the level of each of the N measured biomarkers is indicative of an improvement in the prognosis of the disease or disorder in the subject.

The invention also provides a method for monitoring the rate of decline or rate of improvement of the susceptibility to develop the disease or disorder in a subject, comprising a method for monitoring levels of biomarkers in a subject prior to the onset of the disease or disorder as described herein, comprising the additional step of dividing the increase or decrease of biomarker levels by the time interval between the taking of the first (i.e. previous) and second samples from the subject.

Furthermore, the invention provides a method for monitoring the rate of decline or rate of improvement of prognosis of the disease or disorder in a subject, comprising a method for monitoring levels of biomarkers in a subject with a disease or disorder as described herein, comprising the additional step of dividing the increase or decrease of biomarker levels by the time interval between the taking of the first (i.e. previous) and second samples from the subject.

A further aspect of the invention provides a method of measuring the effectiveness of a medicament which has been administered to a subject for the prophylactic treatment of a disease or disorder or to treat a disease or disorder in that subject, the method comprising:

(i) measuring the levels of N biomarkers in a biological sample obtained from the subject treated with the medicament, wherein N is 2-13;
(ii) comparing the measured levels of the N biomarkers with the corresponding levels of the biomarkers in a biological sample obtained from a control;
wherein the N biomarkers are selected from the group consisting of:

| | |
|---|---|
| EM1 | Phenylalanine (Phe) |
| EM2 | Tyrosine (Tyr) |
| EM3 | Isoleucine (Ile) |
| EM4 | Leucine (Leu) |
| EM5 | Ornithine (Orn) |
| EM6 | Proline (Pro) |
| EM7 | Glutamate (Glu) |
| EM8 | Glycine (Gly) |
| EM9 | Glutamine (Gln) |
| EM10 | Methionine (Met) |
| EM11 | Aspartate (Asp) |
| EM12 | Valine (Val) |
| EM13 | Arginine (Arg) |

wherein differences in the levels of the N measured biomarkers in the biological sample obtained from the treated subject compared to the corresponding levels of biomarkers in the biological sample obtained from the control provide an indication of the efficacy of the medicament in that subject.

A further aspect of the invention provides a method of measuring the effectiveness of a medicament which has been administered to a subject for the prophylactic treatment of a disease or disorder or to treat a disease or disorder in that subject, the method comprising:

(i) measuring the levels of N biomarkers in a biological sample obtained from the subject treated with the medicament, wherein N is 2-13;
(ii) comparing the measured levels of the N biomarkers with the corresponding levels of the biomarkers in a biological sample previously obtained from the subject;
wherein the N biomarkers are selected from the group consisting of:

| | |
|---|---|
| EM1 | Phenylalanine (Phe) |
| EM2 | Tyrosine (Tyr) |
| EM3 | Isoleucine (Ile) |
| EM4 | Leucine (Leu) |
| EM5 | Ornithine (Orn) |
| EM6 | Proline (Pro) |
| EM7 | Glutamate (Glu) |
| EM8 | Glycine (Gly) |
| EM9 | Glutamine (Gln) |
| EM10 | Methionine (Met) |
| EM11 | Aspartate (Asp) |
| EM12 | Valine (Val) |
| EM13 | Arginine (Arg) |

wherein differences in the levels of the N measured biomarkers in the biological sample obtained from the treated subject compared to the corresponding levels of biomarkers in the biological sample previously obtained from the subject provide an indication of the efficacy of the medicament in that subject.

In particular, an increase in the level of each of the N measured biomarkers is indicative of the medicament being (prophylactically) efficacious.

Furthermore, a decrease in the level of each of the N measured biomarkers is indicative of a lack of (prophylactic) efficacy of the medicament.

The invention also provides a method for monitoring the effect of the medicament on the rate of decline or rate of improvement of susceptibility to develop a disease or disorder as defined herein in a subject, comprising a method for monitoring levels of biomarkers in a subject prior to onset of a disease or disorder as described herein, comprising the additional step of dividing the increase or decrease of biomarker levels by the time interval between the taking of the first (i.e. previous) and second samples from the subject.

The invention also provides a method for monitoring the effectiveness of a medicament which has been administered to a subject to treat a disease or disorder as described above, which comprises the additional step of administering the medicament to the subject prior to step (i) or in the interval between the taking of samples.

In the context of the above methods for monitoring the effectiveness of a medicament, in some embodiments of the invention, the medicament has been administered to the subject before the two biological samples have been obtained. In other embodiments, the medicament has been administered to the subject in the interval between the taking of the two samples.

In some preferred embodiments, N is 3-13, 4-13, 5-13, 6-13, 7-13, 8-13, 9-13, 10-13, 11-13 or 12-13. In other preferred embodiments, N is 2-5, 3-5 or 4-5. In yet other preferred embodiments, N is 2, 3, 4, 5, 6, 7, 8, 9, 10, 11, 12 or 13. In yet others the endogenous metabolites are selected from EMs 1-2, 1-3, 4-4, 1-5, 1-6, 1-7, 1-8, 1-9, 1-10, 1-11, 1-12 or 1-13.

In a further embodiment of the invention, there is provided a method of preventing, delaying or reducing the onset of, or treating earlier than hitherto possible, a disturbed metabolic profile in a subject, which comprises:

a) detecting a disturbed metabolic profile caused by a disease or disorder as defined herein, by a method comprising:
(i) measuring the levels of N endogenous metabolites in a biological sample obtained from the subject, wherein N is 2-13, in order to produce a metabolic profile of the N endogenous metabolites in that subject;
(ii) comparing the measured levels of the N endogenous metabolites with the corresponding levels of the endogenous metabolites in a biological sample obtained from a control;
wherein the N endogenous metabolites are selected from the group consisting of:

| | |
|---|---|
| EM1 | Phenylalanine (Phe) |
| EM2 | Tyrosine (Tyr) |
| EM3 | Isoleucine (Ile) |
| EM4 | Leucine (Leu) |
| EM5 | Ornithine (Orn) |
| EM6 | Proline (Pro) |
| EM7 | Glutamate (Glu) |
| EM8 | Glycine (Gly) |
| EM9 | Glutamine (Gln) |
| EM10 | Methionine (Met) |
| EM11 | Aspartate (Asp) |
| EM12 | Valine (Val) |
| EM13 | Arginine (Arg) |

wherein the disturbance in the metabolic profile is one wherein there is independently a decrease or increase in the level of each of the N measured endogenous metabolites in the biological sample obtained from the subject compared to the corresponding levels of endogenous metabolites in the biological sample obtained from the control; and wherein the disturbance in the metabolic profile is due to one or more of the diseases or disorders defined herein; and
(b1) prescribing or supplying to the subject or recommending treatment of the subject with an amount of N-(2-chloro-3,4-dimethoxybenzylideneamino)guanidine or an aminoguanidine, either as the free base or in salt form, effective to prevent, delay, reduce or treat the onset of said disease or disorder, or
(b2) administering to said subject an amount of N-(2-chloro-3,4-dimethoxybenzylideneamino) guanidine or an aminoguanidine, either as the free base or in salt form, effective to prevent, delay, reduce or treat the onset of said disease or disorder.

Also provided is a method of preventing, delaying or reducing the onset of, or treating earlier than hitherto possible, a disturbed metabolic profile in a subject, which comprises:

(a) detecting a disturbed metabolic profile caused by a disease or disorder as defined herein, by a method comprising:
(i) measuring the levels of N endogenous metabolites in a biological sample obtained from the subject, wherein N is 2-13, in order to produce a metabolic profile of the N endogenous metabolites in that subject;
(ii) comparing the measured levels of the N endogenous metabolites with the corresponding levels of the endogenous metabolites in a biological sample previously obtained from the subject;
wherein the N endogenous metabolites are selected from the group consisting of:

| | |
|---|---|
| EM1 | Phenylalanine (Phe) |
| EM2 | Tyrosine (Tyr) |
| EM3 | Isoleucine (Ile) |
| EM4 | Leucine (Leu) |
| EM5 | Ornithine (Orn) |
| EM6 | Proline (Pro) |
| EM7 | Glutamate (Glu) |
| EM8 | Glycine (Gly) |
| EM9 | Glutamine (Gln) |
| EM10 | Methionine (Met) |
| EM11 | Aspartate (Asp) |
| EM12 | Valine (Val) |
| EM13 | Arginine (Arg) |

wherein the disturbance in the metabolic profile is one wherein there is independently a decrease or increase in the level of each of the N measured endogenous metabolites in the biological sample obtained from the subject compared to the corresponding levels of endogenous metabolites in the biological sample previously obtained from the subject; and wherein the disturbance in the metabolic profile is due to one or more of the diseases or disorders as defined above; and
(b1) prescribing or supplying to the subject or recommending treatment of the subject with an amount of N-(2-chloro-3,4-dimethoxybenzylideneamino)guanidine or an aminoguanidine, either as the free base or in salt form, effective to prevent, delay, reduce or treat the onset of said disease or disorder, or
(b2) administering to said subject an amount of N-(2-chloro-3,4-dimethoxybenzylideneamino) guanidine or an aminoguanidine, either as the free base or in salt form, effective to prevent, delay, reduce or treat the onset of said disease or disorder.

In a further embodiment, the invention provides a method for preventing, delaying or reducing the onset of, or for treating earlier than hitherto possible, a disease or disorder (as hereinbelow defined) in a subject, which comprises

a) detection of an elevated risk for, diagnosis and prognosis of a disease or disorder in a subject, by a method comprising:
(i) measuring the levels of N biomarkers in a biological sample obtained from the subject, wherein N is 2-13;
(ii) comparing the measured levels of the N biomarkers with the corresponding levels of the biomarkers in a biological sample obtained from a control;
wherein the N biomarkers are selected from the group consisting of:

| | |
|---|---|
| EM1 | Phenylalanine (Phe) |
| EM2 | Tyrosine (Tyr) |
| EM3 | Isoleucine (Ile) |
| EM4 | Leucine (Leu) |
| EM5 | Ornithine (Orn) |
| EM6 | Proline (Pro) |
| EM7 | Glutamate (Glu) |
| EM8 | Glycine (Gly) |
| EM9 | Glutamine (Gln) |
| EM10 | Methionine (Met) |
| EM11 | Aspartate (Asp) |
| EM12 | Valine (Val) |
| EM13 | Arginine (Arg) |

wherein a decrease in the level of each of the N measured biomarkers in the biological sample obtained from the subject compared to the corresponding levels of biomarkers in the biological sample obtained from the control provides an indication of an elevated risk for, a diagnosis of or reduced prognosis of a disease or disorder in the subject, and b) then administering to said subject an amount of N-(2-chloro-3,4-dimethoxybenzylideneamino)guanidine, either as the free base or in salt form, effective to prevent, delay, reduce or treat the onset of said disease or disorder.

In another embodiment, the invention provides a method for preventing, delaying or reducing the onset of, or for treating earlier than hitherto possible, a disease or disorder (as hereinbelow defined) in a subject, which comprises

a) detection of an elevated risk for, diagnosis and prognosis of a disease or disorder in a subject, by a method comprising:
(i) measuring the levels of N biomarkers in a biological sample obtained from the subject, wherein N is 2-13;
(ii) comparing the measured levels of the N biomarkers with the corresponding levels of the biomarkers in a biological sample previously obtained from the subject;
wherein the N biomarkers are selected from the group consisting of:

| | |
|---|---|
| EM1 | Phenylalanine (Phe) |
| EM2 | Tyrosine (Tyr) |
| EM3 | Isoleucine (Ile) |
| EM4 | Leucine (Leu) |
| EM5 | Ornithine (Orn) |
| EM6 | Proline (Pro) |
| EM7 | Glutamate (Glu) |
| EM8 | Glycine (Gly) |
| EM9 | Glutamine (Gln) |
| EM10 | Methionine (Met) |
| EM11 | Aspartate (Asp) |
| EM12 | Valine (Val) |
| EM13 | Arginine (Arg) |

wherein a decrease in the level of each of the N, measured biomarkers in the biological sample obtained from the subject compared to the corresponding levels of biomarkers in the biological sample previously obtained from the subject is indicative of an elevated risk for, a diagnosis of or reduced prognosis of the disease or disorder in the subject, and b) then administering to said subject an amount of N-(2-chloro-3,4-dimethoxybenzylideneamino)guanidine, either as the free base or in salt form, effective to prevent, delay, reduce or treat the onset of said disease or disorder.

In some embodiments of the invention, EM6 proline (Pro) is replaced by 4-hydroxyproline. In other embodiments, EM6 is the combined level of proline and 4-hydroxyproline in the relevant sample.

As used in this context, the term “disease or disorder” refers to:

(a) all autoimmune diseases;
(b) all autoimmune diseases or disorders which are characterised by tissue degradation, for example myelin or connective tissue in e.g. joints and skin;
(c) all autoimmune diseases or disorders which are characterised by degradation of connective tissue, for example in the joints, skin and eyes;
(d) all autoimmune diseases or disorders which are characterised by degradation of myelin in the central nervous system;
(e) all autoimmune diseases or disorders which affect the skin by degradation, for example of connective tissue or hardening of the skin;
(f) all autoimmune diseases or disorders which affect the digestive system, for example by degradation of bowel tissue;
(g) all autoimmune diseases or disorders which affect the cardiovascular system, for example by degradation of blood vessels;
(h) all autoimmune diseases or disorders which affect the neurological system, for example by causing inflammatory lesions in the brain;
(i) all autoimmune diseases or disorders which affect the kidneys and/or lungs, for example by causing inflammatory destruction of blood vessels in these organs;
(j) all diabetic disorders, for example T1D and T2D;
(k) all arthritic disorders, for example OA and RA; or

### (l) T1D, RA and OA.

Examples of the diseases or disorders referred to in (b)-(i) may be found above in the section headed “Groups and subgroups” and in the corresponding diseases mentioned in Table 1.

In some embodiments, a decrease in the level of each of the N measured biomarkers is indicative of the diagnosis of the disease or disorder in the subject.

In some embodiments, a decrease in the level of each of the N measured biomarkers is indicative of a decline in the prognosis of the disease or disorder in the subject.

In some embodiments, an increase in the level of each of the N measured biomarkers is indicative of an improvement in the prognosis of the disease or disorder in the subject.

The invention also provides a method for monitoring the rate of decline or rate of improvement of the susceptibility to develop the disease or disorder in a subject, comprising a method for monitoring levels of biomarkers in a subject prior to the onset of the disease or disorder as described herein, comprising the additional step of dividing the increase or decrease of biomarker levels by the time interval between the taking of the first (i.e. previous) and second samples from the subject.

Furthermore, the invention provides a method for monitoring the rate of decline or rate of improvement of prognosis of the disease or disorder in a subject, comprising a method for monitoring levels of biomarkers in a subject with a disease or disorder as described herein, comprising the additional step of dividing the increase or decrease of biomarker levels by the time interval between the taking of the first (i.e. previous) and second samples from the subject.

A further aspect of the invention provides a method of measuring the effectiveness of a medicament which has been administered to a subject for the prophylactic treatment of a disease or disorder or to treat a disease or disorder in that subject, the method comprising:

(i) measuring the levels of N biomarkers in a biological sample obtained from the subject treated with the medicament, wherein N is 2-13;
(ii) comparing the measured levels of the N biomarkers with the corresponding levels of the biomarkers in a biological sample obtained from a control;
wherein the N biomarkers are selected from the group consisting of:

| | |
|---|---|
| EM1 | Phenylalanine (Phe) |
| EM2 | Tyrosine (Tyr) |
| EM3 | Isoleucine (Ile) |
| EM4 | Leucine (Leu) |
| EM5 | Ornithine (Orn) |
| EM6 | Proline (Pro) |
| EM7 | Glutamate (Glu) |
| EM8 | Glycine (Gly) |
| EM9 | Glutamine (Gln) |
| EM10 | Methionine (Met) |
| EM11 | Aspartate (Asp) |
| EM12 | Valine (Val) |
| EM13 | Arginine (Arg) |

wherein differences in the levels of the N measured biomarkers in the biological sample obtained from the treated subject compared to the corresponding levels of biomarkers in the biological sample obtained from the control provide an indication of the efficacy of the medicament in that subject.

A further aspect of the invention provides a method of measuring the effectiveness of a medicament which has been administered to a subject for the prophylactic treatment of a disease or disorder or to treat a disease or disorder in that subject, the method comprising:

(i) measuring the levels of N biomarkers in a biological sample obtained from the subject treated with the medicament, wherein N is 2-13;
(ii) comparing the measured levels of the N biomarkers with the corresponding levels of the biomarkers in a biological sample previously obtained from the subject;
wherein the N biomarkers are selected from the group consisting of:

| | |
|---|---|
| EM1 | Phenylalanine (Phe) |
| EM2 | Tyrosine (Tyr) |
| EM3 | Isoleucine (Ile) |
| EM4 | Leucine (Leu) |
| EM5 | Ornithine (Orn) |
| EM6 | Proline (Pro) |
| EM7 | Glutamate (Glu) |
| EM8 | Glycine (Gly) |
| EM9 | Glutamine (Gln) |
| EM10 | Methionine (Met) |
| EM11 | Aspartate (Asp) |
| EM12 | Valine (Val) |
| EM13 | Arginine (Arg) |

wherein differences in the levels of the N measured biomarkers in the biological sample obtained from the treated subject compared to the corresponding levels of biomarkers in the biological sample previously obtained from the subject provide an indication of the efficacy of the medicament in that subject.

In particular, an increase in the level of each of the N measured biomarkers is indicative of the medicament being (prophylactically) efficacious.

Furthermore, a decrease in the level of each of the N measured biomarkers is indicative of a lack of (prophylactic) efficacy of the medicament.

In some embodiments, N is preferably N is 3-13, 4-13, 5-13, 6-13, 7-13, 8-13, 9-13, 10-13, 11-13 or 12-13. In other embodiments, N is preferably 2-5, 3-5 or 4-5. In yet other embodiments N is preferably N is 2, 3, 4, 5, 6, 7, 8, 9, 10, 11, 12 or 13. In still yet other embodiments, the endogenous metabolites are selected from EMs 1-2, 1-3, 1-4, 1-5, 1-6, 1-7, 1-8, 1-9, 1-10, 1-11, 1-12 or 1-13.

In a further aspect of the invention, there is provided a method of preventing, delaying or reducing the onset of, or treating earlier than hitherto possible, a disturbed metabolic profile in a subject, which comprises:

a) detecting a disturbed metabolic profile caused by rheumatoid arthritis, by a method comprising:
(i) measuring the levels of N endogenous metabolites in a biological sample obtained from the subject, wherein N is 2-11, in order to produce a metabolic profile of the N endogenous metabolites in that subject;
(ii) comparing the measured levels of the N endogenous, metabolites with the corresponding levels of the endogenous metabolites in a biological sample obtained from a control;
wherein the N endogenous metabolites are selected from the group consisting of:

| | |
|---|---|
| EM1 | Phenylalanine |
| EM2 | Tyrosine |
| EM3 | Isoleucine |
| EM8 | Glycine |
| EM9 | Glutamine |
| EM10 | Methionine |
| EM14 | Lysine |
| EM15 | Asparagine |
| EM16 | Serine |
| EM17 | Tryptophan |
| EM18 | Threonine |

wherein the disturbance in the metabolic profile is one wherein there is independently a decrease or increase in the level of each of the N measured endogenous metabolites in the biological sample obtained from the subject compared to the corresponding levels of endogenous metabolites in the biological sample obtained from the control;
and wherein the disturbance in the metabolic profile is due to rheumatoid arthritis; and
(b1) prescribing or supplying to the subject or recommending treatment of the subject with an amount of N-(2-chloro-3,4-dimethoxybenzylideneamino)guanidine or an aminoguanidine, either as the free base or in salt form, effective to prevent, delay, reduce or treat the onset of rheumatoid arthritis, or
(b2) administering to said subject an amount of N-(2-chloro-3,4-dimethoxybenzylideneamino) guanidine or an aminoguanidine, either as the free base or in salt form, effective to prevent, delay, reduce or treat the onset of rheumatoid arthritis.

Also provided is a method of preventing, delaying or reducing the onset of, or treating earlier than hitherto possible, a disturbed metabolic profile in a subject, which comprises:

(a) detecting a disturbed metabolic profile caused by rheumatoid arthritis, by a method comprising:
(i) measuring the levels of N endogenous metabolites in a biological sample obtained from the subject, wherein N is 2-11, in order to produce a metabolic profile of the N endogenous metabolites in that subject;
(ii) comparing the measured levels of the N endogenous metabolites with the corresponding levels of the endogenous metabolites in a biological sample previously obtained from the subject;
wherein the N endogenous metabolites are selected from the group consisting of:

| | |
|---|---|
| EM1 | Phenylalanine |
| EM2 | Tyrosine |
| EM3 | Isoleucine |
| EM8 | Glycine |
| EM9 | Glutamine |
| EM10 | Methionine |
| EM14 | Lysine |
| EM15 | Asparagine |
| EM16 | Serine |
| EM17 | Tryptophan |
| EM18 | Threonine |

wherein the disturbance in the metabolic profile is one wherein there is independently a decrease or increase in the level of each of the N measured endogenous metabolites in the biological sample obtained from the subject compared to the corresponding levels of endogenous metabolites in the biological sample previously obtained from the subject; and wherein the disturbance in the metabolic profile is due to rheumatoid arthritis; and
(b1) prescribing or supplying to the subject or recommending treatment of the subject with an amount of N-(2-chloro-3,4-dimethoxybenzylideneamino)guanidine or an aminoguanidine, either as the free base or in salt form, effective to prevent, delay, reduce or treat the onset of rheumatoid arthritis, or
(b2) administering to said subject an amount of N-(2-chloro-3,4-dimethoxybenzylideneamino) guanidine or an aminoguanidine, either as the free base or in salt form, effective to prevent, delay, reduce or treat the onset of rheumatoid arthritis.

In some preferred embodiments N is 3-11, 4-11, 5-11, 6-11, 7-11, 8-11, 9-11 or 10-11. In other preferred embodiments, N is 2-5, 3-5 or 4-5. In yet other preferred embodiments, N is 2, 3, 4, 5, 6, 7, 8, 9, 10 or 11. In still other preferred embodiments, the endogenous metabolites are selected from EMs 1-3 and/or EMs 8-10 and/or EMs 14-18; or the endogenous metabolites are selected from EM2, EM3, EM8 and EM10.

The term “measuring” includes assaying, quantifying, imaging or otherwise establishing the presence or absence of the target endogenous metabolite profile, subunits thereof, or combinations of reagent bound targets, and the like, or assaying for, imaging, ascertaining, establishing, or otherwise determining one or more factual characteristics of a disease or disorder as defined herein.

The levels of the N individual endogenous metabolites in the biological samples may be measured using the same or different techniques.

The present invention relates, inter alia, to a method for diagnosis and/or detecting a disease or disorder as defined herein in a subject comprising detecting a endogenous metabolite profile in a biological sample from the subject. The endogenous metabolite profile may be measured using any suitable means. For example, the endogenous metabolite profile may be measured using chromatographic and/or spectroscopic methods or a reagent that detects or binds to the endogenous metabolites in the profile, preferably using individual antibodies specifically reactive with the individual endogenous metabolites of the profile or a part thereof.

In one embodiment, a set of endogenous metabolites has now been found that identifies subjects at risk of developing certain diseases or disorders. A subject's profile of these endogenous metabolites may therefore aid in the prognosis, detection and diagnosis of those diseases or disorders. The profile of endogenous metabolites may also be used as a tool for screening and identification of drugs and new chemical entities (NCEs) which act to restore normal levels of these endogenous metabolites, thereby preventing or delaying the onset of these diseases or disorders and thus being efficacious in the treatment of those diseases or disorders.

The invention also provides a method for monitoring the effect of the medicament on the rate of decline or rate of improvement of susceptibility to develop certain diseases or disorders in a subject, comprising a method for monitoring levels of endogenous metabolites in a subject prior to onset of those diseases or disorders as described herein, comprising the additional step of dividing the increase or decrease of endogenous metabolite levels by the time interval between the taking of the first (i.e. previous) and second samples from the subject.

The invention also provides a method for monitoring the effectiveness of a medicament which has been administered to a subject to treat certain diseases or disorders as described above, which comprises the additional step of administering the medicament to the subject prior to step (i) or in the interval between the taking of samples.

A set of endogenous metabolites has now been found that identifies subjects at risk of developing T1D and/or T2D. A subject's profile of these endogenous metabolites may therefore aid in the prognosis, detection and diagnosis of T1D and/or T2D. The profile of endogenous metabolites may also be used as a tool for screening and identification of drugs and new chemical entities (NCEs) which act to restore normal levels of these endogenous metabolites, thereby preventing or delaying the onset of the T1D and/or T2D and thus being efficacious in the treatment of T1D and/or T2D.

The compound N-(2-chloro-3,4-dimethoxybenzylideneamino)guanidine, used herein either as the free base or in salt form, e.g. as the acetate, m.pt. 198-200° C., is a known compound. Its preparation and formulations suitable for containing it have been described in, e.g., WO 02/11715. The invention includes the use of any precursors or pro-drugs thereof. In the context of this disclosure, this compound is only to be used in combination with the methods of the invention (i.e. detection, preventing, etc.).

The invention further provides the compound N-(2-chloro-3,4-dimethoxybenzylideneamino) guanidine, either as the free base or in salt form, or a precursor or pro-drug therefor, for use in preventing, reducing or delaying the onset of autoimmune disease in a subject who has been shown (tested) by the use of endogenous metabolite analysis as disclosed herein to be at elevated risk, or perhaps even in the very early stages, of such disease. Such analysis can be carried out before any of the more obvious symptoms of AD have presented themselves, and will enable prophylaxis or treatment to be commenced at a far earlier stage than has hitherto been possible as a result of existing forms of diagnosis.

Examples of suitable chromatographic and spectroscopic methods include Fourier Transform Infra-Red (FT-IR) spectroscopy, nuclear magnetic resonance (NMR) spectroscopy, high-performance liquid chromatography (HPLC), liquid chromatography-mass spectrometry (LC-MS) and gas chromatography-mass spectrometry (GC-MS).

In one embodiment, the invention relates to a method for the measurement of the endogenous metabolite profile by a chromatography-based technique. The endogenous metabolite compounds can be detected as they are or they can be chemically transformed (derivatized) before detection. Methods of detection can be, but are not limited to, light absorbance of the sample, preferably a UV-detector, or by measuring the mass/charge ratio by a mass spectrometer (e.g. Fonteh, A. N. et al., Amino Acids. 32: 203-212 (2007)).

Other methods for measuring endogenous metabolites include spectroscopic techniques used in conjunction with chemometric methods, e.g. principal component analysis (PCA), partial least squares projections to latent structures (PLS), orthogonal PLS (OPLS), PLS discriminant analysis (PLS-DA) and orthogonal PLS-DA (OPLS-DA).

Other methods for measuring endogenous metabolites include taking a multi-wavelength spectroscopy measurement, typically a transmission spectrum of the sample in e.g. the near-infrared range of the electromagnetic spectrum and comparing the spectrum with a standard sample spectrum from a control subject. From a comparison based on chemometric methods it is then determined whether the sample indicates a disease or disorder as defined herein.

In a preferred embodiment of the invention, the levels of one or more of the N endogenous metabolites are independently measured using reagents which individually bind to the individual endogenous metabolites, and which are then detected.

Preferably, the levels of one or more of the N endogenous metabolites are measured using individual antibodies which are specific for the N endogenous metabolites.

The antibodies may be directly or indirectly labelled using a detectable label. Examples of detectable labels include enzymes. Preferably, the substrate for the enzyme is selected so that the substrate, or a reaction product of the enzyme and substrate, forms a fluorescent complex; and the level of the endogenous metabolite is measured by measuring the level of the fluorescent complex.

The antibodies specific for the endogenous metabolite profile used in the methods of the invention may be obtained from scientific or commercial sources. Alternatively, isolated native constituents of the endogenous metabolite profile or recombinant constituents of the endogenous metabolite profile may be utilized to prepare antibodies, monoclonal or polyclonal antibodies, and immunologically active fragments (e.g. a Fab or (Fab)₂ fragment), an antibody heavy chain, an antibody light chain, humanized antibodies, a genetically engineered single chain Fv molecule (Ladner et al, U.S. Pat. No. 4,946,778). Antibodies including monoclonal and polyclonal antibodies, fragments and chimeras, may be prepared using methods known to those skilled in the art.

Antibodies specifically reactive with the endogenous metabolite profile, or derivatives thereof, may be used to detect the endogenous metabolite profile in various biological samples, for example they may be used in any known immunoassays which rely on the binding interaction between an antigenic determinant of a protein and the antibodies. Examples of such assays are radioimmunoassays, enzyme immunoassays (e.g. ELISA), immunofluorescence, immunoprecipitation, latex agglutination, hemagglutination, and histochemical tests.

The sample, or antibodies specific for the endogenous metabolite profile, may be immobilized. Examples of suitable carriers are agarose, cellulose, dextran, Sephadex, Sepharose, liposomes, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, well, beads, disc, sphere etc. The immobilized antibody may be prepared by reacting the material with a suitable insoluble carrier using known chemical or physical methods, for example, cyanogen bromide coupling.

In accordance with an embodiment, the present invention provides means for determining the endogenous metabolite profile in a sample by measuring the endogenous metabolite profile immunoassay. A variety of immunoassay methods can be used to measure the endogenous metabolite profile. In general, an immunoassay method for the detection of the endogenous metabolite profile may be competitive or non-competitive. Competitive methods typically employ immobilized or immobilizable antibodies to the endogenous metabolite profile and labelled forms of the endogenous metabolite profile. Sample of the endogenous metabolite profile and labelled constituents of the endogenous metabolite profile compete for binding to antibodies specific for the constituents of the endogenous metabolite profile. After separation of the resulting labelled endogenous metabolite profile constituents that has become bound to antibodies specific for the endogenous metabolite profile (bound fraction) from that which has remained unbound (unbound fraction), the amount of the label in either bound or unbound fraction is measured and may be correlated with the amount of the endogenous metabolite profile in the test sample in any conventional manner, e.g., by comparison to a standard curve.

The above-described immunoassay methods and formats are intended to be exemplary and are not limiting since, in general, it will be understood that any immunoassay method or format can be used in the present invention.

The terms “sample”, “biological sample”, and the like mean a material known to or suspected of containing or expressing the endogenous metabolites in the profile. The test sample can be used directly as obtained from the source or following a pre-treatment to modify the character of the sample. The sample can be derived from any biological source, such as tissues or extracts, including cells, and physiological fluids, such as, for example, whole blood, plasma, serum; saliva, ocular lens fluid, cerebrospinal fluid, sweat, urine, milk, ascites fluid, synovial fluid, peritoneal fluid and the like. The sample can be obtained from animals, preferably mammals, most preferably humans. The sample can be treated prior to use, such as preparing plasma from blood, diluting viscous fluids, and the like. Methods of treatment can involve filtration, distillation, extraction, concentration, inactivation of interfering components, the addition of reagents, and the like. In a preferred embodiment, the biological sample is a biological fluid, more preferably blood or synovial fluid.

The term “subject” refers to a warm-blooded animal such as a mammal which is afflicted with, or suspected to be afflicted with a disease or disorder as defined herein. Preferably, the subject is a human.

As used herein, the term “control” relates to an individual or group of individuals of the same species as the subject being tested. For example, if the subject is a human, the control will also be a human.

The “control” will generally be a group of one or more individuals who show no signs of exhibiting symptoms of a disease or disorder as defined herein. In particular, the controls may be individuals or groups of individuals who are considerably younger than the subject to be tested (e.g. individuals under the age of 30, 40, 50 or 60) or the controls may be unaffected genetic relations who may be age-matched.

In some embodiments of the invention, the control or control group is of the same age or approximately the same age as the subject.

The endogenous metabolite levels in the control may, for example, be available from published charts, computer databases, look-up tables, etc. In other embodiments, the term encompasses a level which has previously been determined. Thus the method of the invention is not limited to methods which comprise the step of physically testing the level of endogenous metabolite obtained from a control.

Levels for control samples from healthy subjects may be established by prospective and/or retrospective statistical studies. Healthy subjects who have no clinically evident disease or abnormalities may be selected for statistical studies. Diagnosis may be made by a finding of statistically different levels of endogenous metabolite profile compared to a control sample or previous levels quantified for the same subject.

Preferably, the changes in the levels of the endogenous metabolites (between the subject and the control samples or between the samples taken at different time intervals from the subject) are significant changes.

In some embodiments of the present invention, a significant increase is one where the level of measured endogenous metabolite in the biological sample obtained from the subject is more than a 10%, 20%, 30%, 40%, 50%, 60%, 70%, 80%, 90% or 100% increase compared to the corresponding level in the biological sample obtained from a control. In other embodiments of the invention, a significant increase means that the increase is significant using the criteria p<0.05, 2-tailed test.

In other embodiments of the present invention, a significant decrease is one where the level of measured endogenous metabolite in the biological sample obtained from the subject is more than a 10%, 20%, 30%, 40%, 50%, 60%, 70%, 80%, 90% or 100% decrease compared to the corresponding level in the biological sample obtained from a control. In other embodiments of the invention, a significant decrease means that the decrease is significant using the criteria p<0.05, 2-tailed lest.

In some embodiments of the methods disclosed herein, the method additionally comprises the step of obtaining a biological sample directly from the subject and/or directly from one or more controls.

In yet other embodiments of the invention, the method additionally comprises the step of administering to the subject a medicament, preferably one which is appropriate for the diagnosis, i.e. a medicament for the treatment of a disease or disorder as defined herein.

The methods of the invention will in general be carried out in vitro or ex vivo. In particular, the term “biological sample obtained from” a subject or control is intended to indicate that the methods are not carried out on the human or animal body. In particular, the term “obtained from” may comprise receiving a sample from an agent acting on behalf of the subject, e.g. receiving a sample from a doctor, nurse, hospital, medical centre, etc., either directly or indirectly, e.g. via a courier or postal service.

In the methods of the invention where the levels of endogenous metabolites in a biological sample are compared to the corresponding levels in a biological sample previously obtained from a subject, the time interval between the taking of samples may be any appropriate period, e.g. 1-60 months, 1-36 months, 1-12 months or 1-6 months.

The methods of the invention may be carried out using a diagnostic kit for quantifying the endogenous metabolite profile in a biological sample. The invention also relates to kits for carrying out the methods of the invention.

In particular, the invention provides a kit for use in a method as defined herein, comprising reagents for detecting the presence of N endogenous metabolites selected from the group consisting of:

| | |
|---|---|
| EM1 | Phenylalanine (Phe) |
| EM2 | Tyrosine (Tyr) |
| EM3 | Isoleucine (Ile) |
| EM4 | Leucine (Leu) |
| EM5 | Ornithine (Orn) |
| EM6 | Proline (Pro) |
| EM7 | Glutamate (Glu) |
| EM8 | Glycine (Gly) |
| EM9 | Glutamine (Gln) |
| EM10 | Methionine (Met) |
| EM11 | Aspartate (Asp) |
| EM12 | Valine (Val) |
| EM13 | Arginine (Arg) |

wherein N is 2-13, optionally together with instructions for use. Preferably, N is 3-13, 4-13, 5-13, 6-13, 7-13, 8-13, 9-13, 10-13, 11-13, 12-13 or 13.

The invention also provides a kit for use in a method as defined herein, comprising reagents for detecting the presence of N endogenous metabolites selected from the group consisting of:

| | |
|---|---|
| EM1 | Phenylalanine |
| EM2 | Tyrosine |
| EM3 | Isoleucine |
| EM8 | Glycine |
| EM9 | Glutamine |
| EM10 | Methionine |
| EM14 | Lysine |
| EM15 | Asparagine |
| EM16 | Serine |
| EM17 | Tryptophan |
| EM18 | Threonine |

wherein N is 2-11, optionally together with instructions for use. Preferably, N is 3-11, 4-11, 5-11, 6-11, 7-11, 8-11, 9-11, 10-11 or 11.

Also provided is the use of a kit of the invention for the diagnosis or detection of a disease or disorder as defined herein.

By way of example, the kit may contain antibodies specific for the N endogenous metabolites, antibodies against the antibodies labelled with an enzyme; and a substrate for the enzyme. The kit may also contain one or more of microtiter plate wells, standards, assay diluent, wash buffer, adhesive plate covers, and/or instructions for carrying out a method of the invention using the kit.

An embodiment of the invention would then consist of a kit containing a solution for extraction of N endogenous metabolites, a suitable system of eluents, suitable internal or external standards and possibly a suitable chromatographic column and possibly reagents for derivatization of the endogenous metabolite compounds. One example of a commercially available technique for this quantification is the HPLC Amino Acid Analysis Solution from Waters Corporation (Waters, Corporation).

### BRIEF DESCRIPTION OF THE FIGURES

FIG. 1. Natural history of type 1 diabetes. TRIGR is a current prevention trial aiming at preventing the development of islet autoimmunity. DPT-1 and ENDIT are past clinical trials in islet autoantibody positive subjects with predictable risk for type 1 diabetes. Metabolomics analyses of serum may define changes due to initiators before the appearance of islet autoimmunity or to promoters inducing hyperglycemia i.e. the onset of clinical diabetes.

FIG. 2. Four stages of evolution of inflammatory arthritis, adapted from (Dixon, W. G. et al., Best Pract. Res. Clin. Rheumatol. 19: 37-53 (2005)). During the first stage leading up to the onset of symptoms., the combination of genetic and environmental factors triggers an autoimmune response and the development of joint inflammation in the patient. This phase may share many common features for all ADs. In the second stage persistence of the inflammatory polyarthritis (IP) is determined. Again, for reasons largely unknown, for some patients the joint inflammation is temporary and some patients develop a chronic inflammation. In the third and fourth phases the type of arthritis and severity of the disease is determined. A large proportion of patients who develop an acute IP will make a complete recovery within days or a few weeks, often without even consulting the primary care unit.

FIG. 3A. Scores scatter plot (t[1]) from an OPLS-DA model of human controls and RA patients. The subjects with RA (upper part of the plot, highlighted with triangles) are well separated from the controls (lower part of the plot, highlighted with box symbols). This separation is entirely based on the patterns of endogenous metabolites of the two groups of individuals.

FIG. 3B. Loadings column plot (p[1], correlation scaled) from an OPLS-DA model of human controls and RA patients. From this plot the metabolite profile that describes the observed separation between controls and patients diagnosed with RA can be identified. Negative loadings denote endogenous metabolites with decreased levels in subjects with an RA diagnosis. All amino acids are down regulated in the RA group.

FIG. 4A. Scores scatter plot (t[1]/to [1]) from an OPLS-DA model of human controls and OA patients. The subjects with OA (right side in the plot, highlighted with triangles) are well separated from the controls (left side in the plot, highlighted with box symbols). This separation is entirely based on the patterns of endogenous metabolites of the two groups of individuals.

FIG. 4B. Loadings column plot (p[1], correlation scaled) from an OPLS-DA model of human controls and OA patients. From this plot the metabolite profile that describes the observed separation between controls and patients diagnosed with OA can be identified. Negative loadings denote endogenous metabolites with decreased levels in subjects with an OA diagnosis. All amino acids are down regulated in the OA group.

FIG. 5A. Scores scatter plot (t[1]/to [1]) from an OPLS-DA model of CIA mice and healthy controls. The mice with CIA (right side in the plot, highlighted with triangles) are well separated from the controls (left side in the plot, highlighted with box symbols). This separation is entirely based on the patterns of endogenous metabolites of the two groups of individuals.

FIG. 5B. Loadings column plot (p[1], correlation scaled) from an OPLS-DA model of CIA mice and healthy controls. From this plot the metabolite profile that describes the observed separation between controls and disease group can be identified. Negative loadings denote endogenous metabolites with decreased levels in subjects with CIA. All metabolites are down regulated in the disease group.

FIG. 6A. Scores scatter plot (t[1]/to [1]) from an OPLS-DA model of AIA rats and healthy controls. The AIA rats (right side in the plot, highlighted with triangles) are well separated from the controls (left side in the plot, highlighted with box symbols).

FIG. 6B. Loadings column plot (p[1], correlation scaled) from an OPLS-DA model of AIA rats and healthy controls. Many metabolites are down regulated in the disease group.

FIG. 7A. Scores scatter plot (t[1]/to [1]) from an OPLS-DA model of AIA rats treated with an experimental substance. The untreated AIA animals (right side in the plot, highlighted with triangles) are well separated from the AIA animals who have received treatment with the experimental substance (left side in the plot, highlighted with squares). This separation is entirely based on the patterns of endogenous metabolites of the two groups of individuals.

FIG. 7B. Loadings column plot (p[1], correlation scaled) from an OPLS-DA model of AIA rats treated with an experimental substance. Almost all metabolites are up regulated in the treated animals, showing efficacy of the administered medicament.

FIGS. 8A-17A. Scores scatter plot (t[1] vs. to [1]) from OPLS-DA model of various numbers of biomarkers with control subjects vs. subjects with RA. The control samples are highlighted with box symbols and the RA samples are highlighted with triangle symbols. There is excellent separation between healthy controls and diagnosed patients. This separation is entirely based on the patterns of endogenous metabolites of the two groups of individuals.

FIGS. 8B-17B. Plot of correlation scaled loadings from OPLS-DA model of various numbers of biomarkers with control subjects vs. subjects with RA. From this plot the metabolite profile that describes the observed separation between controls and patients diagnosed with RA can be identified. Negative loadings denote biomarkers with decreased levels in subjects with an RA diagnosis.

FIG. 18. Table 5. Summary of results of comparison between the human RA case and relevant animal models. The weights denote the relative importance of the endogenous metabolites and may be used to provide an interpretation of a sample based on a weighted sum of averages.

The present invention is further defined in the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

The disclosure of each reference set forth herein is incorporated herein by reference in its entirety.

### EXAMPLES

### Example 1

Validation of Animal Model as being Relevant for Human ADs.

### Study Design

The human RA and OA samples used consisted of the following blood samples:
19 Control patients
20 RA patients
20 OA patients

Serum samples were collected from 59 persons: 20 of them had the diagnosis RA, 20 of them had the diagnosis OA and 19 blood donors were considered as healthy controls that did not have any diagnosis yet.

The mouse model samples used consist of the following blood samples:
7 samples from controls,
9 samples from subjects with CIA

The rat model samples used consist of the following blood samples:
6 samples from controls
6 samples from subjects with AIA
   Extraction of Metabolites from Serum

Extraction of metabolites from serum samples was essentially performed as the method described by A et al. (2005). 630 μl of MeOH:H₂O (9:1 V:V) including internal standards was added to 70 μl of serum. The solution was vortex mixed for 10 s, kept on ice for 10 min, and then vigorously extracted at a frequency of 30 Hz for 3 min using a MM301 vibration Mill (Retsch GmbH & Co. KG, Haan, Germany. After 120 min on ice, the samples were centrifuged at 19 600 g for 10 min at 4° C. 200 μl of the supernatant was transferred to a GC vial, and 50 μl was transferred to a LC/MS vial and evaporated to dryness.

### GC/TOFMS Analysis

Prior GC/MS analysis the samples were derivatised with 30 μL of methoxyamine hydrochloride (15 mg mL-1) in pyridine by shaking for 10 min at 5° C., then incubating them for 16 h at room temperature. The samples were then trimethylsilylated by adding 30 μL of N-Methyl-N-trifluoroacetamide (MSTFA) with 1% TMCS and incubating them for 1 h at room temperature. After silylation, 30 μL of heptane (containing 0.5 μg methyl stearate as internal standard) was added.

0.1 μl of derivatized sample was injected split less by an Agilent 7683 Series Autosampler (Agilent, Atlanta; GA, USA) into an Agilent 6980 GC equipped with a 10 m×0.18 mm ID, fused silica capillary column chemically bonded with 0.18 μm DB5-MS stationary phase (J&W Scientific, Folsom, Calif., USA). The injector temperature was set at 270° C. Helium was used as carrier gas at a constant flow rate of 1 ml/min through the column. For every analysis, the purge time was set to 60 s at a purge flow rate of 20 ml min-1 and an equilibration time of 1 min. The column temperature was initially kept at 70° C. for 2 min, then increased from 70 to 320° C. at 40° C./min, where it was held for 2 min. The column effluent was introduced into the ion source of a Pegasus III TOFMS (Leco Corp., St Joseph, Mich., USA). The transfer line temperature was set at 250° C. and ion source temperature at 200° C. Ions were generated by a 70 eV electron beam at a current of 2.0 mA. Masses were acquired from m/z 50 to 800 at a rate of 30 spectra/s, and the acceleration voltage was turned on after a solvent delay of 170 s.

Data processing of MS-data To evaluate the extraction protocols, non-processed MS-files from GC/TOFMS and HPLC/MS analysis were exported in NetCDF format to MATLAB software 7.0 (Mathworks, Natick, Mass., USA), where all data-pretreatment procedures, such as base-line correction, chromatogram alignment, time-window setting and multivariate curve resolution (MCR) were performed using custom scripts (Jonsson et al. 2005, 2006). Thus the between sample relative metabolite concentrations were obtained. These data were analysed with partial least squares (PLS) (Wold, S. et al., SIAM J. Sci. Statist. Comput. 5: 735-743 (1984); Hoskuldsson, A., J. Chemometr. 9: 91-123 (1995)) and orthogonal PLS (OPLS) (Trygg, J. et al., J. Chemometrics. 16: 119-128 (2002); Trygg, J. et al., J. Chemometrics. 17: 53-64 (2003)) as implemented in SIMCA-P+ software (Umetrics, AB: (2005)) to identify differences between levels of endogenous metabolites in the samples obtained from the groups of individuals, i.e. human RA and control, CIA mouse and controls, and AIA rat and controls.

### Results

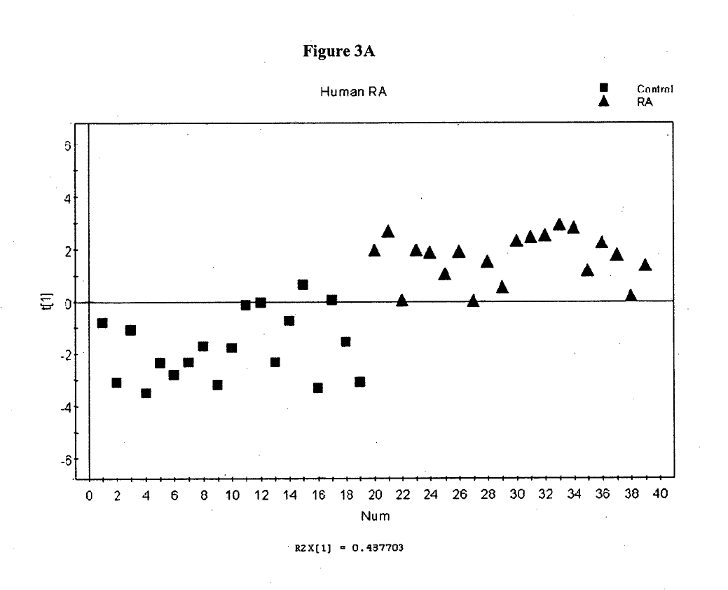

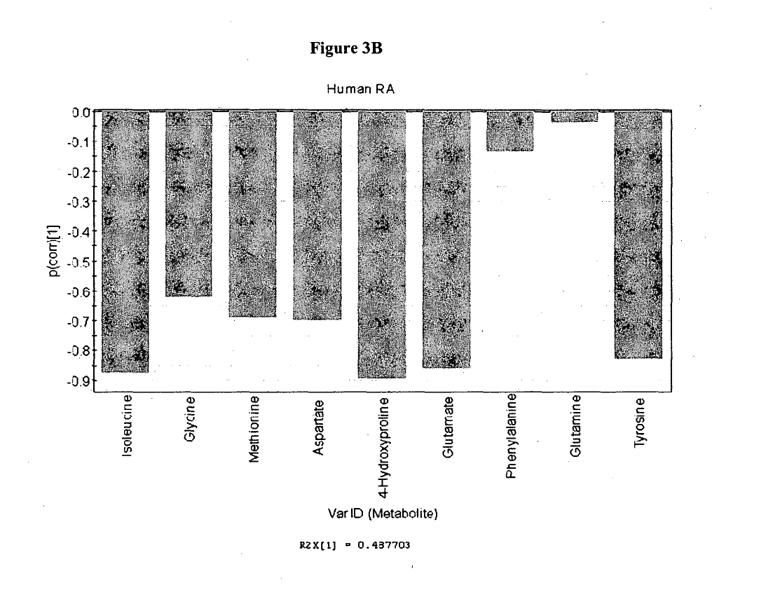

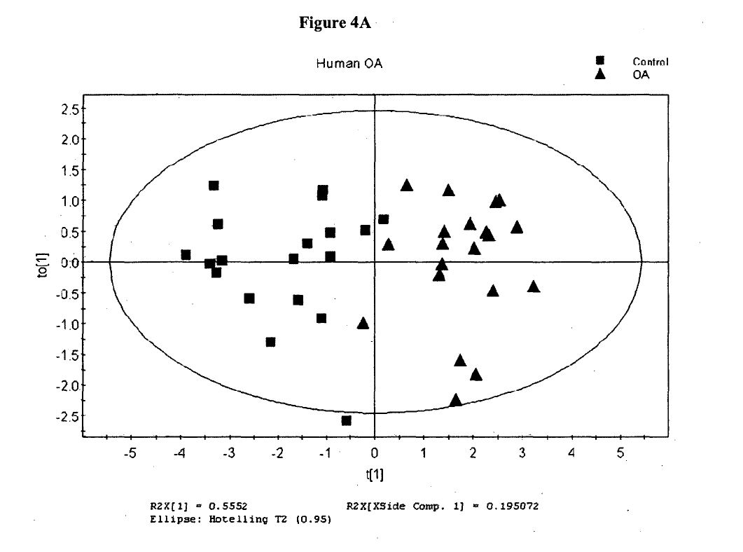

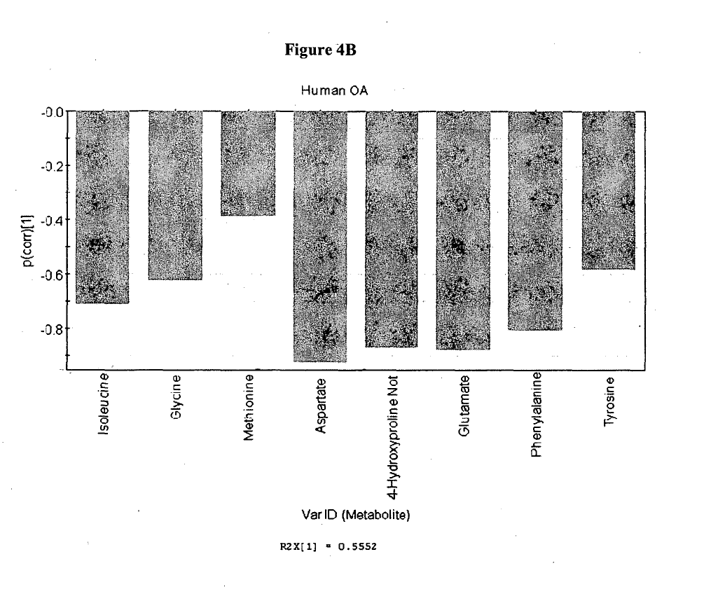

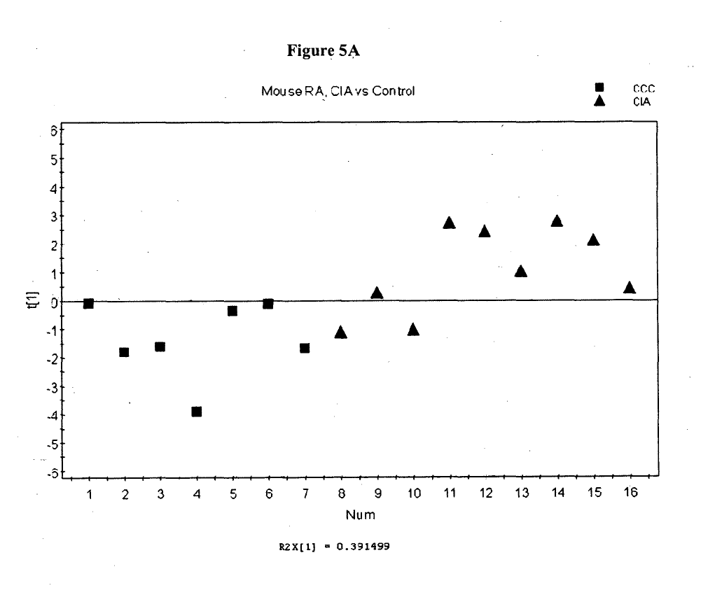

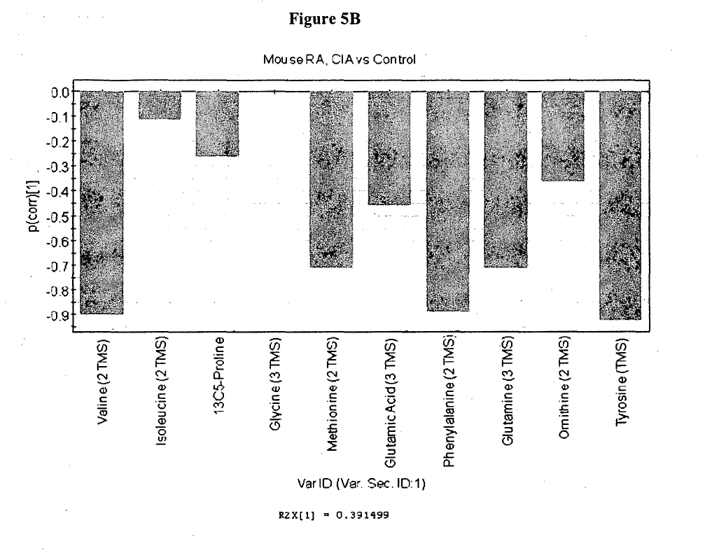

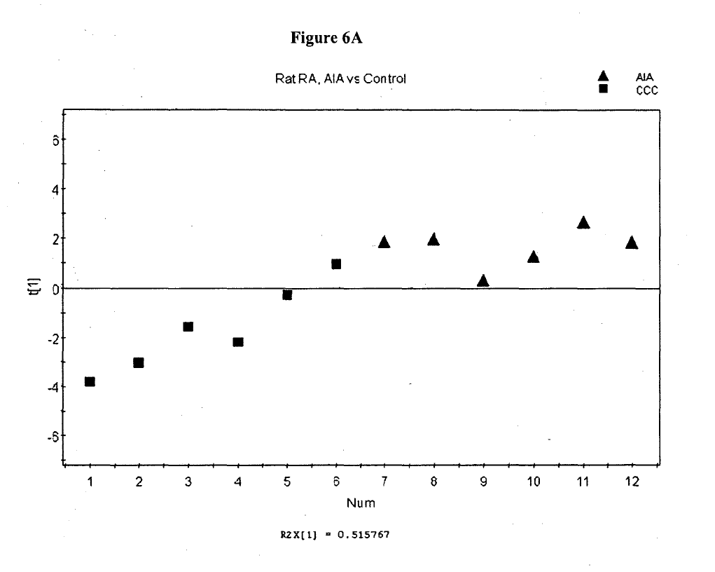

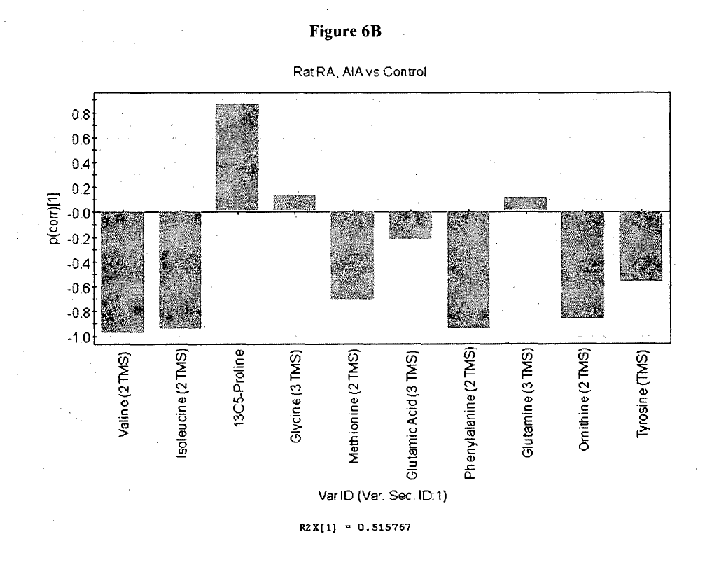

The identified metabolites (Table 5, FIGS. 3A and 3B, 4A and 4B, 5A and 5B, 6A and 6B) consist of amino acids and other metabolites associated with RA and OA, and thus AD in humans. Our OPLS analysis shows that there is a great overlap between the human case and the two animal models and that the metabolites show similar patterns of down regulation in the diseased subjects. The major finding was that it is possible to compare animal models to the human case using this technology and also that it is possible to identify the most relevant model for an indication, in this case AD. This enables validation of the relevance of the animal models for the human case.

### Example 2

### Materials and Methods

The metabolites were extracted from serum according to standard protocols.

### Clinical Samples

The samples used consisted of the following blood samples:
19 Control patients
20 RA patients

### Procedures

Serum samples were collected from 39 persons: 20 of them had the diagnosis RA and 19 blood donors were considered as healthy controls that did not have any diagnosis yet.

The endogenous metabolites were identified by methods well known in the art and between sample relative metabolite concentrations were obtained. These data were analysed with partial least squares (PLS) (Wold, S. et al., SIAM J. Sci. Statist. Comput. 5: 735-743 (1984); Hoskuldsson, A., J. Chemometr. 9: 91-123 (1995)) and orthogonal PLS (OPLS) (Trygg, J. et al., J. Chemometrics. 16: 119-128 (2002); Trygg, J. et al., J. Chemometrics. 17: 53-64 (2003)) as implemented in SIMCA-P+ software (Umetrics, AB: (2005)) to identify differences between levels of endogenous metabolites in the samples obtained from the two groups of individuals.

### Results

The multivariate data analysis revealed a profile of the endogenous metabolites that separate the subjects that have the diagnosis of RA from the healthy-group (FIG. 3A). The result was a very clear separation between the two groups.

Using the model parameters, e.g. loadings or weights, the metabolites that are most potent for diagnosis of RA can be identified (FIG. 3B).

The results of the study show that the diagnostic properties of the endogenous metabolite profile are highly sensitive and specific for the diagnosis of RA in humans. The results also show that diagnosis and monitoring of the disease state are intimately related for a person skilled in the art.

### Example 3

### Materials and Methods

The metabolites were extracted from serum according to standard protocols.

### Clinical Samples

The samples used consisted of the following blood samples:
19 Control patients
20 OA patients

### Procedures

Serum samples were collected from 39 persons: 20 of them had the diagnosis OA and 19 blood donors that were considered as healthy controls that did not have any diagnosis yet.

The endogenous metabolites were identified by methods well known in the art and between sample relative metabolite concentrations were obtained. These data were analysed with partial least squares (PLS) (Wold, S. et al., SIAM J. Sci. Statist. Comput. 5: 735-743 (1984); Hoskuldsson, A., J. Chemometr. 9: 91-123 (1995)) and orthogonal PLS (OPLS) (Trygg, J. et al., J. Chemometrics. 16: 119-128 (2002); Trygg, J. et al., J. Chemometrics. 17: 53-64 (2003)) as implemented in SIMCA-P+ software (Umetrics, AB: (2005)) to identify differences between levels of endogenous metabolites in the samples obtained from the two groups of individuals.

### Results

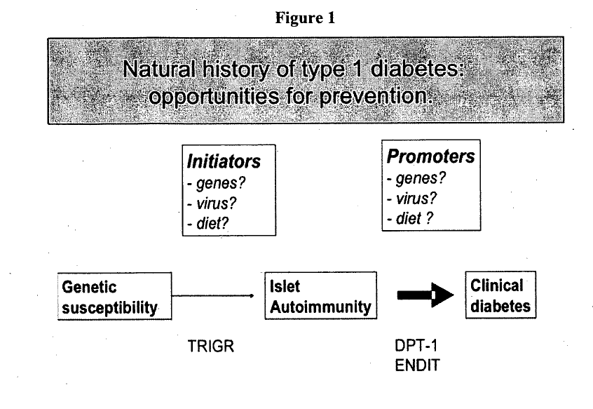

The multivariate data analysis revealed a profile of the endogenous metabolites that separate the subjects that have the diagnosis of OA from the healthy group (FIG. 1). The result was a very clear separation between the two groups.

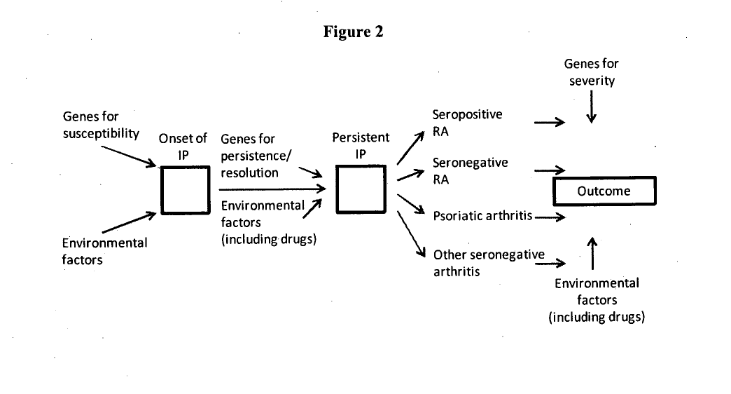

Using the model parameters, e.g. loadings or weights, the metabolites that were most potent for diagnosis of OA were identified (FIG. 2).

The result of the study shows that the diagnostic properties of the endogenous metabolite profile are highly sensitive and specific for the diagnosis of OA in humans. The results also show that diagnosis and monitoring of the disease state are intimately related for a person skilled in the art.

### Example 4

Validation of animal model as being relevant for human RA.

### Study Design

The mouse model samples used consist of the following blood samples:
7 samples from controls
9 samples from subjects with CIA

The rat model samples used consist of the following blood samples:
6 samples from controls
6 samples from subjects with AIA
   Extraction of Metabolites from Serum

Extraction of metabolites from serum samples was essentially performed as the method described by A et al. (2005). 630 μl of MeOH:H₂O (9:1 V:V) including internal standards was added to 70 μl of serum. The solution was vortex mixed for 10 s, kept on ice for 10 min, and then vigorously extracted at a frequency of 30 Hz for 3 min using a MM301 vibration Mill (Retsch GmbH & Co. KG, Haan, Germany. After 120 min on ice, the samples were centrifuged at 19 600 g for 10 min at 4° C. 200 μl of the supernatant was transferred to a GC vial, and 50 μl was transferred to a LC/MS vial and evaporated to dryness.

### GC/TOFMS Analysis

Prior GC/MS analysis the samples were derivatised with 30 μl of methoxyamine hydrochloride (15 mg mL-1) in pyridine by shaking for 10 min at 5° C., then incubating them for 16 h at room temperature. The samples were then trimethylsilylated by adding 30 μL of N-Methyl-N-trifluoroacetamide (MSTFA) with 1% TMCS and incubating them for 1 h at room temperature. After silylation, 30 μl, of heptane (containing 0.5 μg methyl stearate as internal standard) was added. 0.1 μl of derivatized sample was injected split less by an Agilent 7683 Series Autosampler (Agilent, Atlanta; GA, USA) into an Agilent 6980 GC equipped with a 10 m×0.18 mm ID, fused silica capillary column chemically bonded with 0.18 μm DB5-MS stationary phase (J&W Scientific, Folsom, Calif., USA). The injector temperature was set at 270° C. Helium was used as carrier gas at a constant flow rate of 1 ml/min through the column. For every analysis, the purge time was set to 60 s at a purge flow rate of 20 ml min-1 and an equilibration time of 1 min. The column temperature was initially kept at 70° C. for 2 min, then increased from 70 to 320° C. at 40° C./min, where it was held for 2 min. The column effluent was introduced into the ion source of a Pegasus III TOFMS (Leco Corp., St Joseph, Mich., USA). The transfer line temperature was set at 250° C. and ion source temperature at 200° C. Ions were generated by a 70 eV electron beam at a current of 2.0 mA. Masses were acquired from m/z 50 to 800 at a rate of 30 spectra/s, and the acceleration voltage was turned on after a solvent delay of 170 s.

Data processing of MS-data To evaluate the extraction protocols, non-processed MS-files from GC/TOFMS and HPLC/MS analysis were exported in NetCDF format to MATLAB software 7.0 (Mathworks, Natick, Mass., USA), where all data-pretreatment procedures, such as base-line correction, chromatogram alignment, time-window setting and multivariate curve resolution (MCR) were performed using custom scripts (Jonsson et al. 2005, 2006). Thus the between sample relative metabolite concentrations were obtained. These data were analysed with partial least squares (PLS) (Wold, S. et al., SIAM J. Sci. Statist. Comput. 5: 735-743 (1984); Hoskuldsson, A., J. Chemometr. 9: 91-123 (1995)) and orthogonal PLS (OPLS) (Trygg, J. et al., J. Chemometrics. 16: 119-128 (2002); Trygg, J. et al., J. Chemometrics. 17: 53-64 (2003)) as implemented in SIMCA-P+ software (Umetrics, AB: (2005)) to identify differences between levels of endogenous metabolites in the samples obtained from the groups of individuals, i.e. human RA and control, CIA mouse and controls, and AIA rat and controls.

### Results

The identified metabolites (Table 5, FIGS. 3A and 3B, 4A and 4B, 5A and 5B, 6A and 6B) consist of amino acids and other metabolites associated with RA and OA, and thus AD in humans. Our OPLS analysis shows that there is a great overlap between the human case and the two animal models and that the metabolites show similar patterns of down regulation in the diseased subjects. The major finding was that it is possible to compare animal models to the human case using this technology and also that it is possible to identify the most relevant model for an indication, in this case AD. This enables validation of the relevance of the animal models for the human case.

### Example 5

Treatment of RA in an AIA Rat Model with an Experimental Substance (Arginine)

### Study Design

AIA was induced in three Dark Agouti (DA) rats and the rats were then given treatment with an experimental substance.

The rat model samples used consist of the following blood samples:
3 samples from AIA DA rats
4 samples (one of the rats was sampled twice) from AIA DA rats treated with the experimental substance.
   Extraction of Metabolites from Serum

Extraction of metabolites from serum samples was essentially performed as the method described by A et al. (2005). 630 μl of MeOH:H₂O (9:1 V:V) including internal standards was added to 70 μl of serum. The solution was vortex mixed for 10 s, kept on ice for 10 min, and then vigorously extracted at a frequency of 30 Hz for 3 min using a MM301 vibration Mill (Retsch GmbH & Co. KG, Haan, Germany. After 120 min on ice, the samples were centrifuged at 19 600 g for 10 min at 4° C. 200 μl of the supernatant was transferred to a GC vial, and 50 μl was transferred to a LC/MS vial and evaporated to dryness.

### GC/TOFMS Analysis

Prior GC/MS analysis the samples were derivatised with 30 μL of methoxyamine hydrochloride (15 mg mL-1) in pyridine by shaking for 10 min at 5° C., then incubating them for 16 h at room temperature. The samples were then trimethylsilylated by adding 30 μL of N-Methyl-N-trifluoroacetamide (MSTFA) with 1% TMCS and incubating them for 1 h at room temperature. After silylation, 30 μL of heptane (containing 0.5 μg methyl stearate as internal standard) was added. 0.1 μl of derivatized sample was injected split less by an Agilent 7683 Series Autosampler (Agilent, Atlanta; GA, USA) into an Agilent 6980 GC equipped with a 10 m×0.18 mm ID, fused silica capillary column chemically bonded with 0.18 μm DB5-MS stationary phase (J&W Scientific, Folsom, Calif., USA). The injector temperature was set at 270° C. Helium was used as carrier gas at a constant flow rate of 1 ml/min through the column. For every analysis, the purge time was set to 60 s at a purge flow rate of 20 ml min-1 and an equilibration time of 1 min. The column temperature was initially kept at 70° C. for 2 min, then increased from 70 to 320° C. at 40° C./min, where it was held for 2 min. The column effluent was introduced into the ion source of a Pegasus III TOFMS (Leco Corp., St Joseph, Mich., USA). The transfer line temperature was set at 250° C. and ion source temperature at 200° C. Ions were generated by a 70 eV electron beam at a current of 2.0 mA. Masses were acquired from m/z 50 to 800 at a rate of 30 spectra/s, and the acceleration voltage was turned on after a solvent delay of 170 s.

Data processing of MS-data To evaluate the extraction protocols, non-processed MS-files from GC/TOFMS and HPLC/MS analysis were exported in NetCDF format to MATLAB software 7.0 (Mathworks, Natick, Mass., USA), where all data-pretreatment procedures, such as base-line correction, chromatogram alignment, time-window setting and multivariate curve resolution (MCR) were performed using custom scripts (Jonsson et al. 2005, 2006). Thus the between sample relative metabolite concentrations were obtained. These data were analysed with partial least squares (PLS) (Wold, S. et al., SIAM J. Sci. Statist. Comput. 5: 735-743 (1984); Hoskuldsson, A., J. Chemometr. 9: 91-123 (1995)) and orthogonal PLS (OPLS) (Trygg, J. et al., J. Chemometrics. 16: 119-128 (2002); Trygg, J. et al., J. Chemometrics. 17: 53-64 (2003)) as implemented in SIMCA-P+ software (Umetrics, AB: (2005)) to identify differences between levels of endogenous metabolites in the samples obtained from the AIA DA rats and the treated individuals.

### Data Analysis

Data describing the between sample relative metabolite concentrations were obtained. These data were analysed with partial least squares (PLS) (Wold, S. et al., SIAM J. Sci. Statist. Comput. 5: 735-743 (1984); Hoskuldsson, A., J. Chemometr. 9: 91-123 (1995)) and orthogonal PLS (OPLS) (Trygg, J. et al., J. Chemometrics. 16: 119-128 (2002); Trygg, J. et al., J. Chemometrics. 17: 53-64 (2003)) as implemented in SIMCA-P+ software (Umetrics, AB: (2005)) to identify differences between levels of endogenous metabolites in the samples obtained from the groups of individuals, i.e. human RA and control, CIA mouse and controls, and AIA rat and controls.

### Results

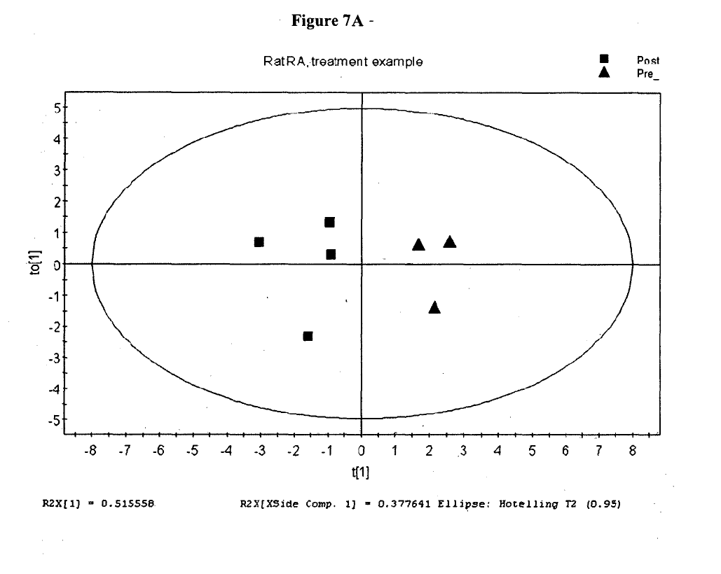

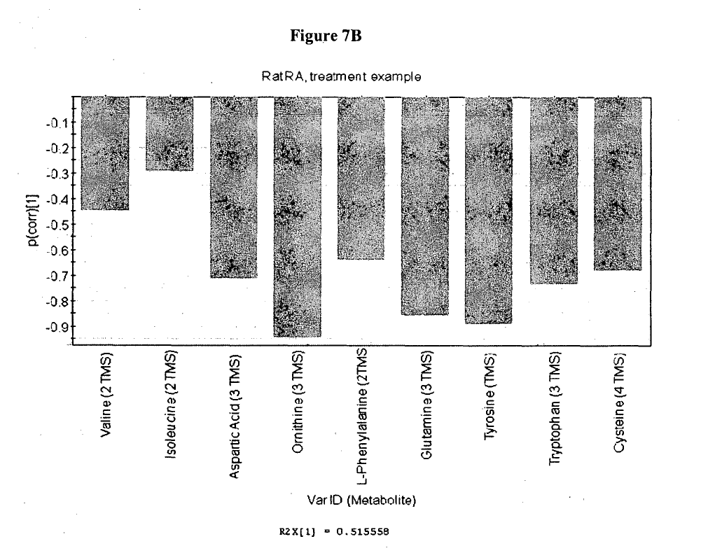

The identified metabolites (FIGS. 7A and 7B) consist of amino acids associated with AD in humans and relevant animal models. Our OPLS analysis shows that the described technology enabled identification of important metabolic changes in the AIA rat after the substance had been administered. The administered substance resulted in up regulation of many of the metabolites associated with AD in humans and relevant animal models. The applied technology for serum analysis of metabolites is sensitive and specific for measuring the efficacy of the administered medicament for the indication AD using this approach.

### Example 6

This example illustrates the potency of the active compound and its therapeutically active acid addition salts for treatment of mental disorders.

### Study Design

AIA was induced in a total of 21 Lewis rats were included in the treatment study. Samples used consist of the following blood samples:
22 samples from AIA Lewis rats (one of the rats was sampled twice)
26 samples from treated AIA Lewis rats (five of the rats was sampled twice)

Different treatments were distributed in the AIA subjects as follows:
5 subjects received 1 mg active compound
5 subjects received 3 mg active compound
6 subjects received 10 mg active compound
5 subjects received 1 mg methotrexate
   Extraction of Metabolites from Serum

Extraction of metabolites from serum samples was essentially performed as the method described by A et al. (2005). 630 μl of MeOH:H₂O (9:1 V:V) including internal standards was added to 70 μl of serum. The solution was vortex mixed for 10 s, kept on ice for 10 min, and then vigorously extracted at a frequency of 30 Hz for 3 min using a MM301 vibration Mill (Retsch GmbH & Co. KG, Haan, Germany. After 120 min on ice, the samples were centrifuged at 19 600 g for 10 min at 4° C. 200 μl of the supernatant was transferred to a GC vial, and 50 μl was transferred to a LC/MS vial and evaporated to dryness.

GC/TOFMS analysis and the Data processing of MS-data was carried out as in Example 1 above.

### Results

The levels of the metabolites, consisting of amino acids and other metabolites associated with T1D, RA and OA, and thus AD in humans, that were identified as relevant endogenous metabolites for detection of risk of AD onset, detection of AD and monitoring of AD progression (Table 5, FIGS. 3A and 3B, 4A and 4B, 5A and 5B, 6A and 6B) were significantly affected by the active compound treatment. Our OPLS analysis shows that the metabolites show similar patterns of up regulation in the subjects treated with the active compound. The major finding was that it is possible to restore levels of metabolites associated with the indication AD in animal models. This has enabled validation of the relevance of the active compound treatment for prophylactic treatment of AD, treatment to delay or prevent onset of AD, and treatment of AD in animal models, and also for the human case.

### Example 7

### Materials and Methods

The metabolites were extracted from serum according to standard protocols.

### Clinical Samples

The samples used consisted of the following blood samples:
19 Control patients
20 RA patients

### Procedures

Serum samples were collected from 39 persons: 20 of them had the diagnosis RA and 19 blood donors were considered as healthy controls that did not have any diagnosis yet.

The endogenous metabolites were identified by methods well known in the art and between sample relative metabolite concentrations were obtained. These data were analysed with partial least squares (PLS) (Wold, S. et al., SIAM J. Sci. Statist. Comput. 5:735-743 (1984); Hoskuldsson, A., J. Chemometr. 9: 91-123 (1995)) and orthogonal PLS (OPLS) (Trygg, J. et al., J. Chemometrics. 16: 119-128 (2002); Trygg, J. et al., J. Chemometrics. 17: 53-64 (2003)) as implemented in SIMCA-P+ software (Umetrics, AB: (2005)) to identify differences between levels of endogenous metabolites in the samples obtained from the two groups of individuals.

### Results

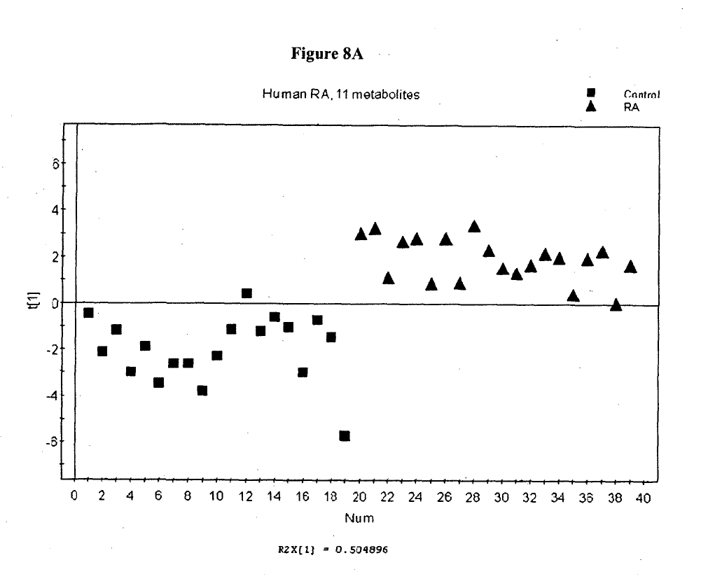

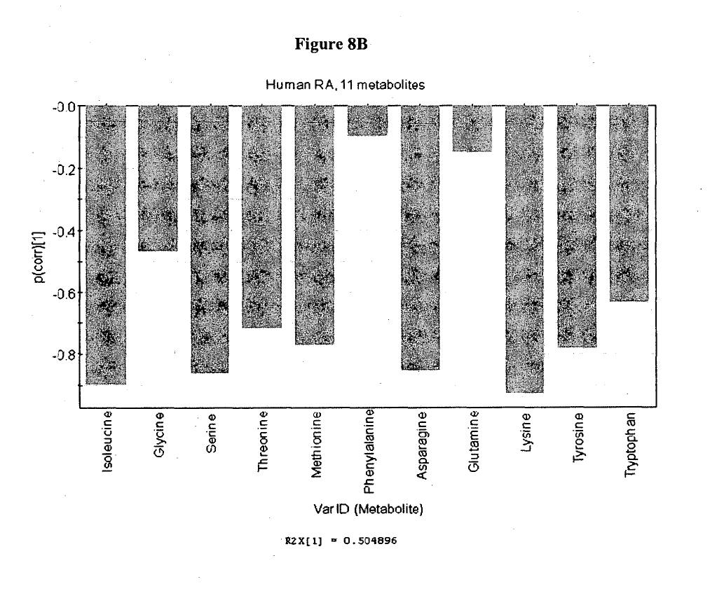

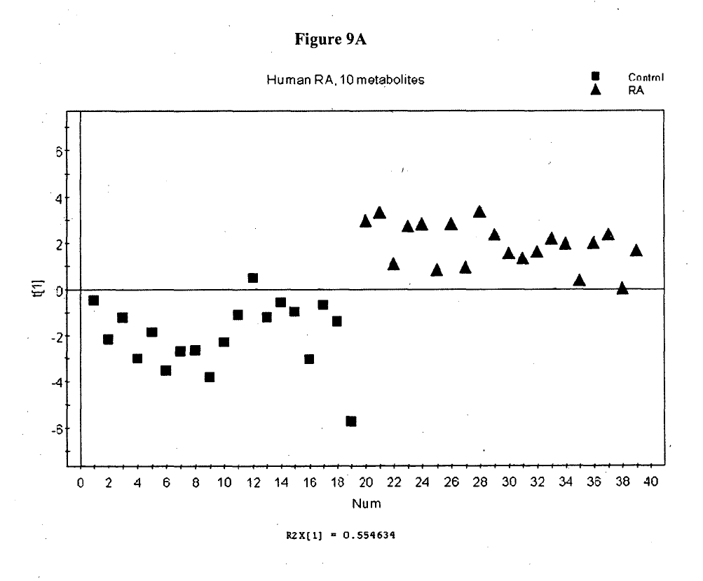

The multivariate data analysis revealed a profile of the endogenous metabolites that separate the subjects that have the diagnosis of RA from the healthy group (FIG. 8A). The result was a very clear separation between the two groups.

Using the model parameters, e.g. loadings or weights, the metabolites that are most potent for diagnosis of RA can be identified (FIG. 8B).

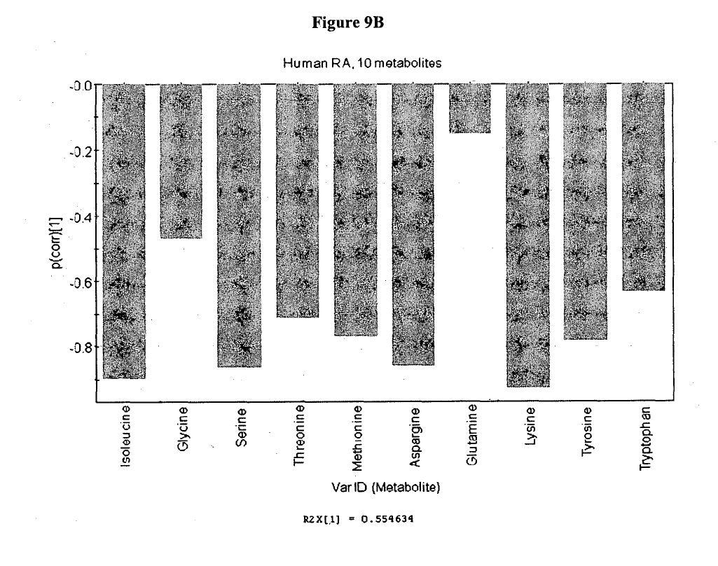

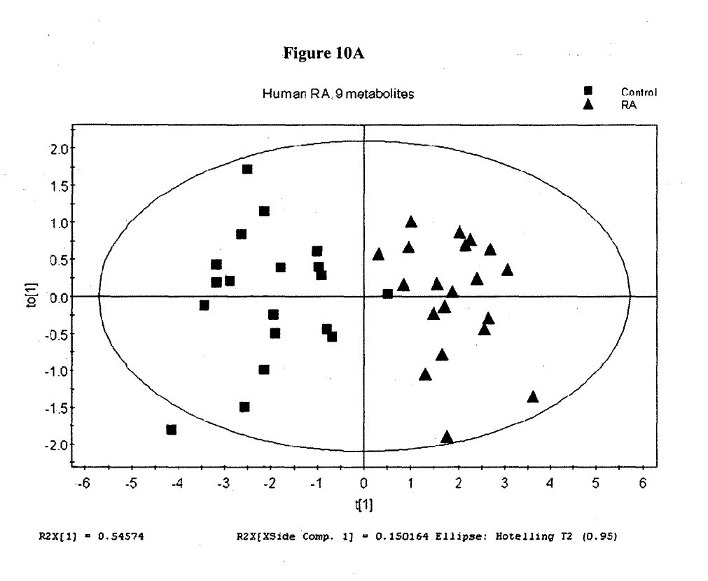

Furthermore, all of the endogenous metabolites do not have to be used to enable separation of subjects that have the diagnosis RA from the healthy group (FIG. 9A). The result was a very clear separation between the two groups. Using the model parameters, e.g. loadings or weights, the metabolites that are most potent for diagnosis of RA can be identified (FIG. 9B).

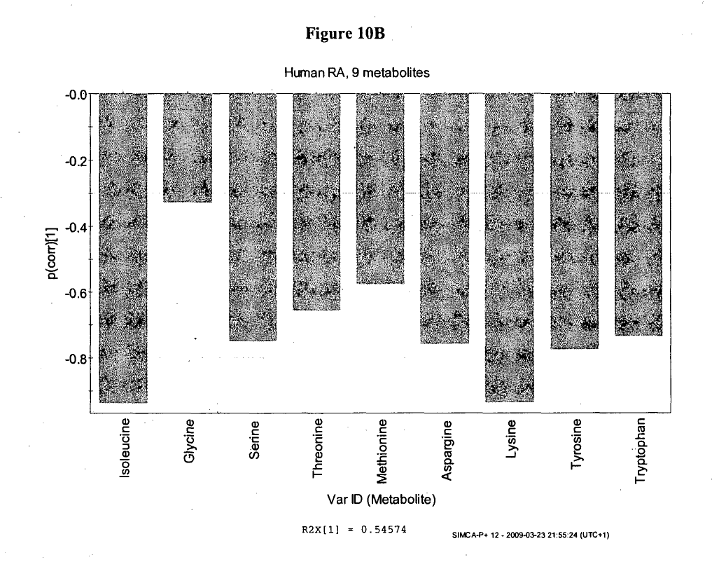

Furthermore, all of the endogenous metabolites do not have to be used to enable separation of subjects that have the diagnosis RA from the healthy group (FIG. 10A). The result was a very clear separation between the two groups. Using the model parameters, e.g. loadings or weights, the metabolites that are most potent for diagnosis of RA can be identified (FIG. 10B).

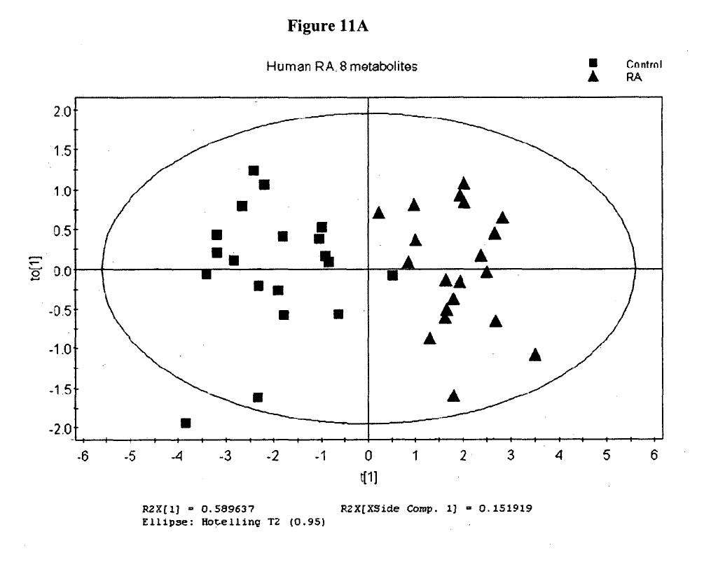

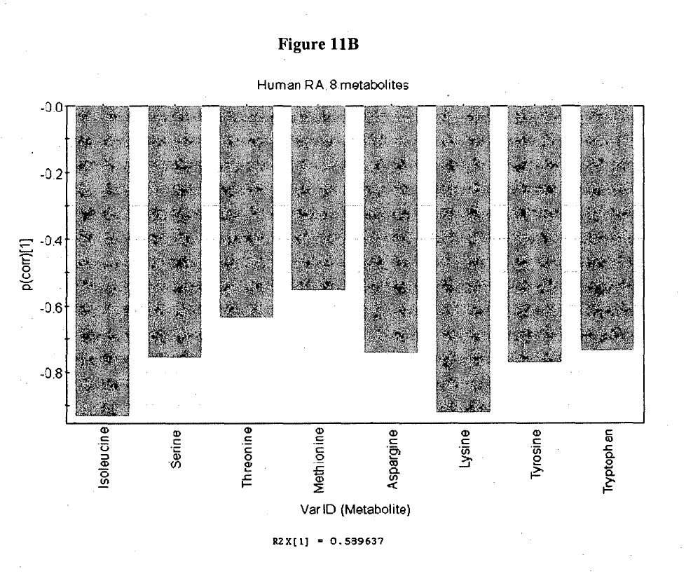

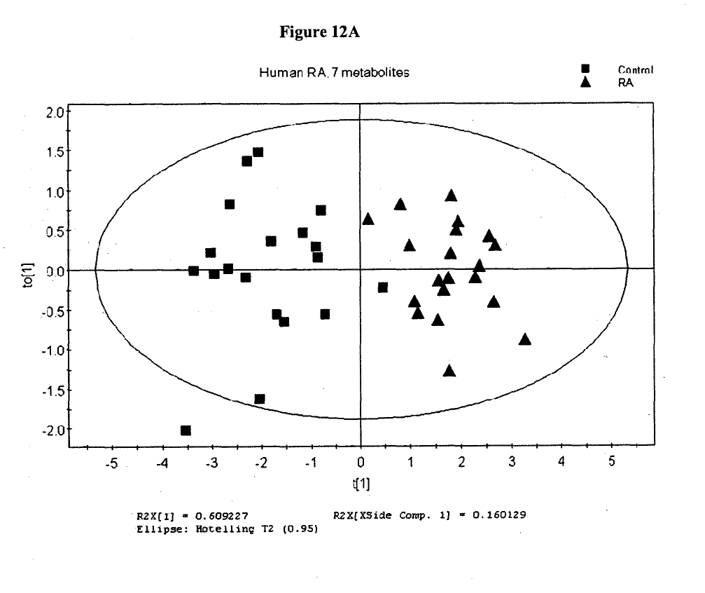

Furthermore, all of the endogenous metabolites do not have to be used to enable separation of subjects that have the diagnosis RA from the healthy group (FIG. 11A). The result was a very clear separation between the two groups. Using the model parameters, e.g. loadings or weights, the metabolites that are most potent for diagnosis of RA can be identified (FIG. 11B).

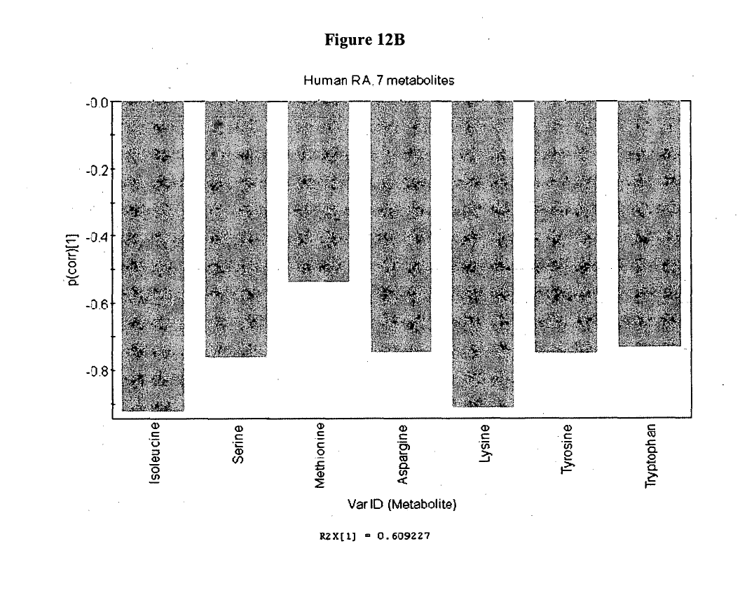

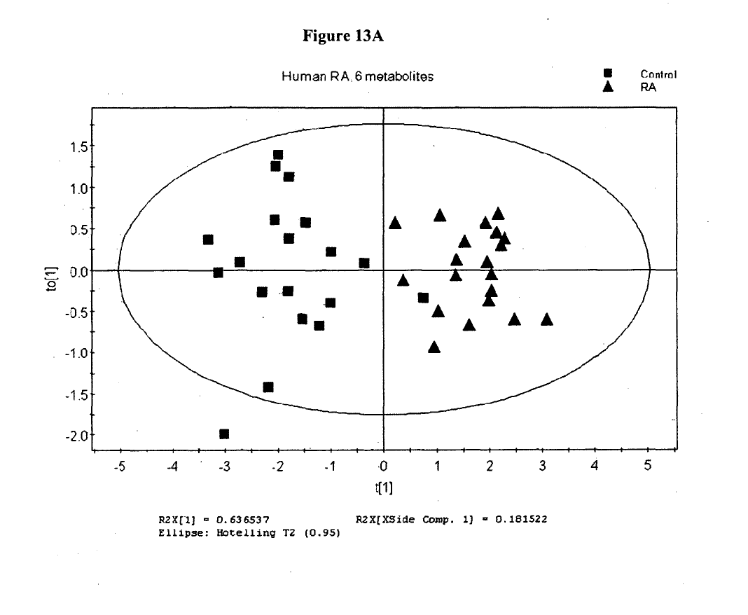

Furthermore, all of the endogenous metabolites do not have to be used to enable separation of subjects that have the diagnosis RA from the healthy group (FIG. 12A). The result was a very clear separation between the two groups. Using the model parameters, e.g. loadings or weights, the metabolites that are most potent for diagnosis of RA can be identified (FIG. 12B).

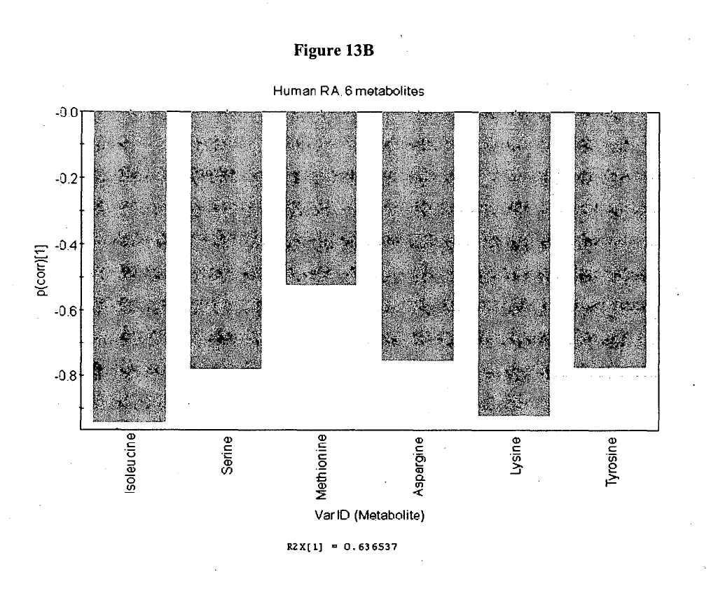

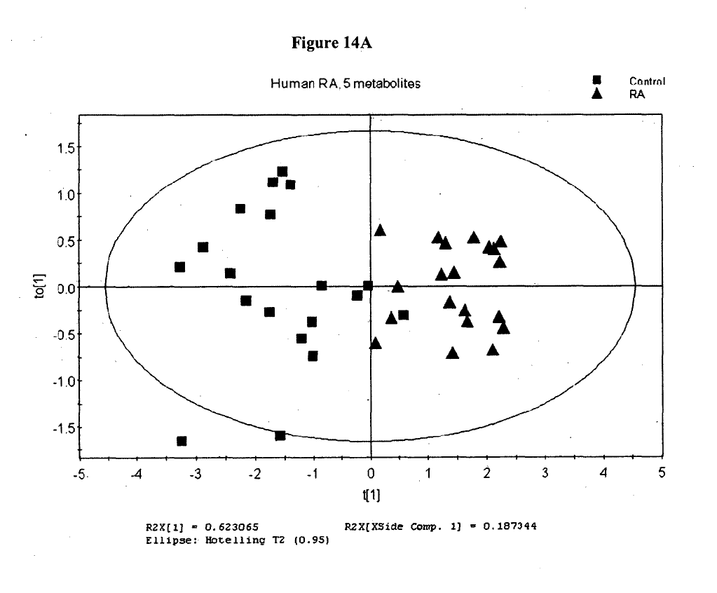

Furthermore, all of the endogenous metabolites do not have to be used to enable separation of subjects that have the diagnosis RA from the healthy group (FIG. 13A). The result was a very clear separation between the two groups. Using the model parameters, e.g. loadings or weights, the metabolites that are most potent for diagnosis of RA can be identified (FIG. 13B).

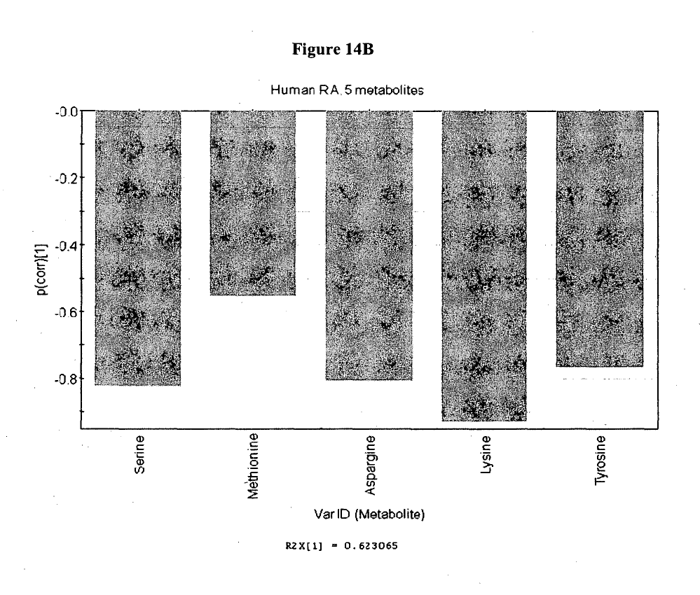

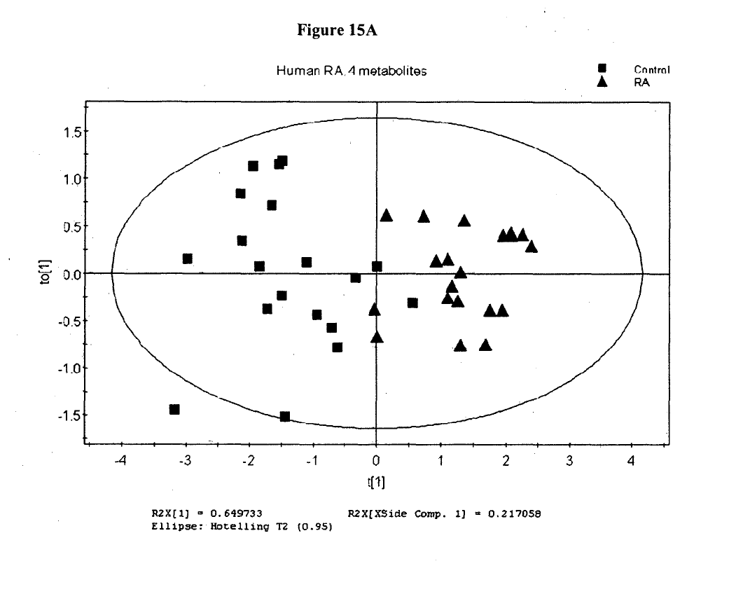

Furthermore, all of the endogenous metabolites do not have to be used to enable separation of subjects that have the diagnosis RA from the healthy group (FIG. 14A). The result was a very clear separation between the two groups. Using the model parameters, e.g. loadings or weights, the metabolites that are most potent for diagnosis of RA can be identified (FIG. 14B).

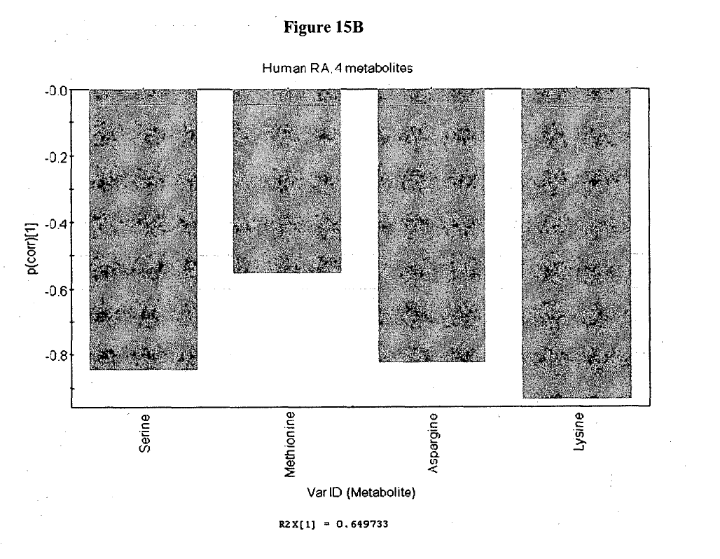

Furthermore, all of the endogenous metabolites do not have to be used to enable separation of subjects that have the diagnosis RA from the healthy group (FIG. 15A). The result was a very clear separation between the two groups. Using the model parameters, e.g. loadings or weights, the metabolites that are most potent for diagnosis of RA can be identified (FIG. 15B).

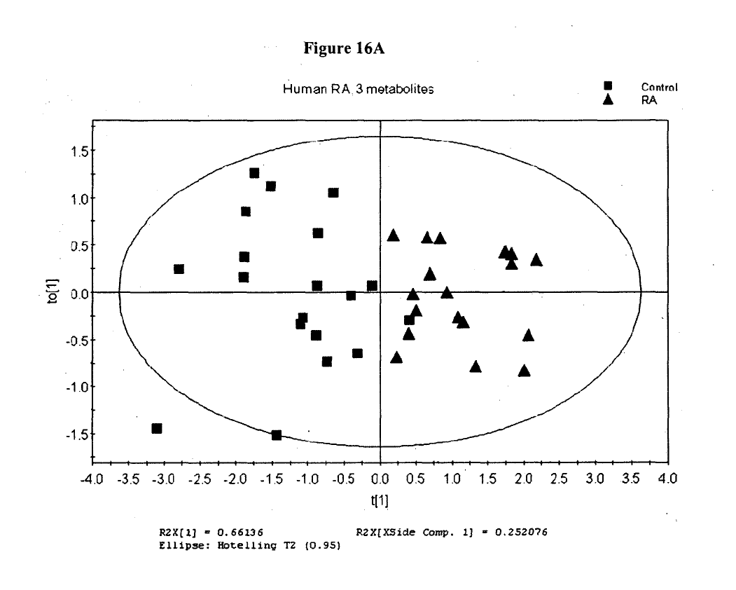

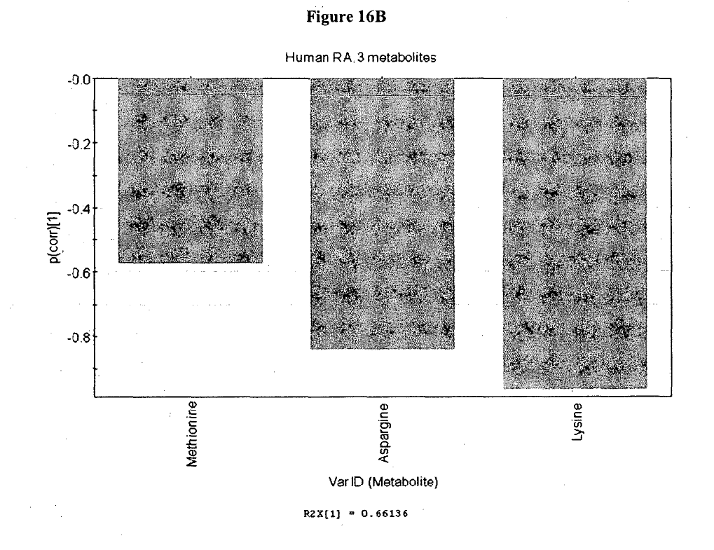

Furthermore, all of the endogenous metabolites do not have to be used to enable separation of subjects that have the diagnosis RA from the healthy group (FIG. 16A). The result was a very clear separation between the two groups. Using the model parameters, e.g. loadings or weights, the metabolites that are most potent for diagnosis of RA can be identified (FIG. 16B).

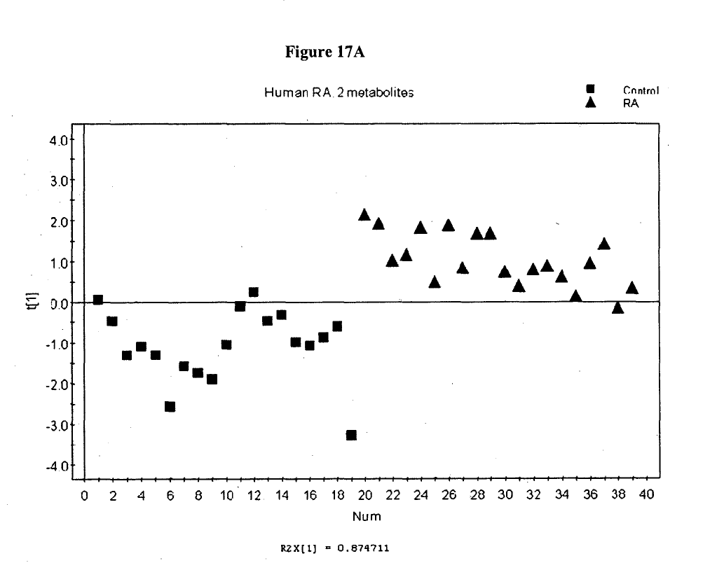

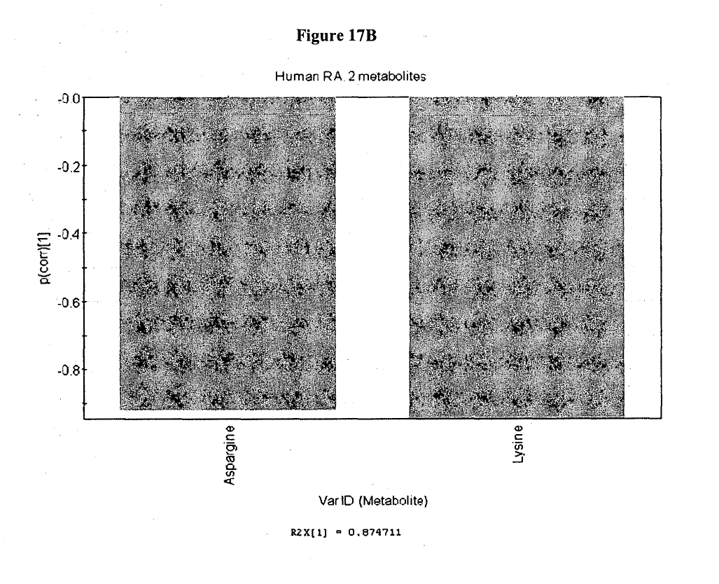

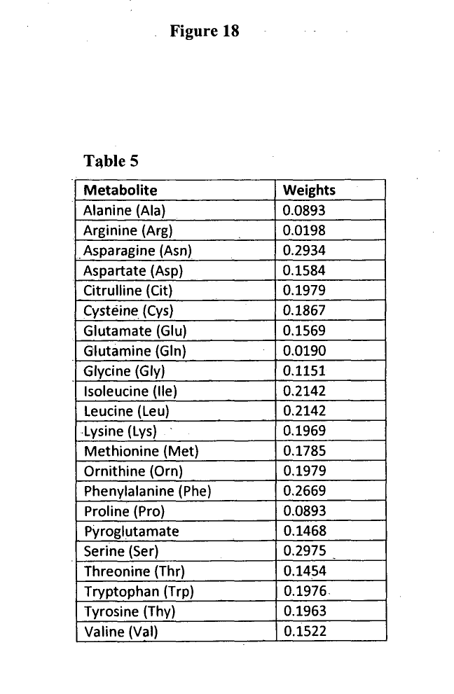

Furthermore, all of the endogenous metabolites do not have to be used to enable separation of subjects that have the diagnosis RA from the healthy group (FIG. 17A). The result was a very clear separation between the two groups. Using the model parameters, e.g. loadings or weights, the metabolites that are most potent for diagnosis of RA can be identified (FIG. 17B).

The results of the study show that the diagnostic properties of the endogenous metabolite profile are highly sensitive and specific for RA in humans. Furthermore, it is the combination of specific endogenous metabolites that offer the enhanced diagnosis properties. The results also show that diagnosis and monitoring of the disease state are intimately related for a person skilled in the art.

1. A method for normalising a disturbance in the metabolic profile of endogenous metabolites in a subject caused by rheumatoid arthritis (RA), the method comprising:
(i) measuring the levels of N endogenous metabolites in a biological sample obtained from the subject, wherein N is 11, in order to produce a metabolic profile of the N endogenous metabolites in that subject;
(ii) comparing the measured levels of the N endogenous metabolites with the corresponding levels of the endogenous metabolites in a biological sample obtained from a control;
wherein the N endogenous metabolites are selected from the group consisting of:

| | |
|---|---|
| EM1 | Phenylalanine |
| EM2 | Tyrosine |
| EM3 | Isoleucine |
| EM8 | Glycine |
| EM9 | Glutamine |
| EM10 | Methionine |
| EM14 | Lysine |
| EM15 | Asparagine |
| EM16 | Serine |
| EM17 | Tryptophan |
| EM18 | Threonine |

wherein the disturbance in the metabolic profile is one wherein there is a decrease or increase in the level of each of the N measured endogenous metabolites in the biological sample obtained from the subject compared to the corresponding levels of endogenous metabolites in the biological sample obtained from the control, and wherein the disturbance in the metabolic profile is due to RA in the subject,
and if a disturbance in the metabolic profile is detected,
(iiia) prescribing or supplying to the subject or recommending treatment of the subject with an effective amount of N-(2-chloro-3,4-dimethoxybenzylideneamino) guanidine or an aminoguanidine, either as the free base or in salt form, for normalising the disturbed metabolic profile;
or
(iiib) administering to said subject an effective amount of N-(2-chloro-3,4-dimethoxybenzylideneamino)guanidine or an aminoguanidine, either as the free base or in salt form, for normalising the disturbed metabolic profile.

2. A method for normalising a disturbance in the metabolic profile of endogenous metabolites in a subject caused by rheumatoid arthritis (RA), the method comprising:
(i) measuring the levels of N endogenous metabolites in a biological sample obtained from the subject, wherein N is 11, in order to produce a metabolic profile of the N endogenous metabolites in that subject;
(ii) comparing the measured levels of the N endogenous metabolites with the corresponding levels of the endogenous metabolites in a biological sample previously obtained from the subject;
wherein the N endogenous metabolites are selected from the group consisting of:

| | |
|---|---|
| EM1 | Phenylalanine |
| EM2 | Tyrosine |
| EM3 | Isoleucine |
| EM8 | Glycine |
| EM9 | Glutamine |
| EM10 | Methionine |
| EM14 | Lysine |
| EM15 | Asparagine |
| EM16 | Serine |
| EM17 | Tryptophan |
| EM18 | Threonine |

wherein the disturbance in the metabolic profile is one wherein there is a decrease or increase in the level of each of the N measured endogenous metabolites in the biological sample obtained from the subject compared to the corresponding levels of endogenous metabolites in the biological sample previously obtained from the subject;
and wherein the disturbance in the metabolic profile is due to RA in the subject,
and if a disturbance in the metabolic profile is detected,
(iiia) prescribing or supplying to the subject or recommending treatment of the subject with an effective amount of N-(2-chloro-3,4-dimethoxybenzylideneamino) guanidine or an aminoguanidine, either as the free base or in salt form, for normalising the disturbed metabolic profile;
or
(iiib) administering to said subject an effective amount of N-(2-chloro-3,4-dimethoxybenzylideneamino)guanidine or an aminoguanidine, either as the free base or in salt form, for normalising the disturbed metabolic profile.

3-20. (canceled)

21. A method of preventing, delaying or reducing the onset of, or treating earlier than hitherto possible, a disturbed metabolic profile in a subject, which comprises:
a) detecting a disturbed metabolic profile caused by rheumatoid arthritis, by a method comprising:
(i) measuring the levels of N endogenous metabolites in a biological sample obtained from the subject, wherein N is 11, in order to produce a metabolic profile of the N endogenous metabolites in that subject;
(ii) comparing the measured levels of the N endogenous metabolites with the corresponding levels of the endogenous metabolites in a biological sample obtained from a control;
wherein the N endogenous metabolites are selected from the group consisting of:

| | |
|---|---|
| EM1 | Phenylalanine |
| EM2 | Tyrosine |
| EM3 | Isoleucine |
| EM8 | Glycine |
| EM9 | Glutamine |
| EM10 | Methionine |
| EM14 | Lysine |
| EM15 | Asparagine |
| EM16 | Serine |
| EM17 | Tryptophan |
| EM18 | Threonine |

wherein the disturbance in the metabolic profile is one wherein there is a decrease or increase in the level of each of the N measured endogenous metabolites in the biological sample obtained from the subject compared to the corresponding levels of endogenous metabolites in the biological sample obtained from the control;
and wherein the disturbance in the metabolic profile is due to rheumatoid arthritis;
and
(b1) prescribing or supplying to the subject or recommending treatment of the subject with an amount of N-(2-chloro-3,4-dimethoxybenzylideneamino)guanidine or an aminoguanidine, either as the free base or in salt form, effective to prevent, delay, reduce or treat the onset of rheumatoid arthritis,
or
(b2) administering to said subject an amount of N-(2-chloro-3,4-dimethoxybenzylideneamino)guanidine or an aminoguanidine, either as the free base or in salt form, effective to prevent, delay, reduce or treat the onset of rheumatoid arthritis.

22. A method of preventing, delaying or reducing the onset of, or treating earlier than hitherto possible, a disturbed metabolic profile in a subject, which comprises:
(a) detecting a disturbed metabolic profile caused by rheumatoid arthritis, by a method comprising:
(i) measuring the levels of N endogenous metabolites in a biological sample obtained from the subject, wherein N is 11, in order to produce a metabolic profile of the N endogenous metabolites in that subject;
(ii) comparing the measured levels of the N endogenous metabolites with the corresponding levels of the endogenous metabolites in a biological sample previously obtained from the subject;
wherein the N endogenous metabolites are selected from the group consisting of:

| | |
|---|---|
| EM1 | Phenylalanine |
| EM2 | Tyrosine |
| EM3 | Isoleucine |
| EM8 | Glycine |
| EM9 | Glutamine |
| EM10 | Methionine |
| EM14 | Lysine |
| EM15 | Asparagine |
| EM16 | Serine |
| EM17 | Tryptophan |
| EM18 | Threonine |

wherein the disturbance in the metabolic profile is one wherein there is a decrease or increase in the level of each of the N measured endogenous metabolites in the biological sample obtained from the subject compared to the corresponding levels of endogenous metabolites in the biological sample previously obtained from the subject;
and wherein the disturbance in the metabolic profile is due to rheumatoid arthritis;
and
(b1) prescribing or supplying to the subject or recommending treatment of the subject with an amount of N-(2-chloro-3,4-dimethoxybenzylideneamino)guanidine or an aminoguanidine, either as the free base or in salt form, effective to prevent, delay, reduce or treat the onset of rheumatoid arthritis,
or
(b2) administering to said subject an amount of N-(2-chloro-3,4-dimethoxybenzylideneamino)guanidine or an aminoguanidine, either as the free base or in salt form, effective to prevent, delay, reduce or treat the onset of rheumatoid arthritis.

23-27. (canceled)

28. A method as claimed in claim 1, wherein the biological sample is blood or synovial fluid.

29. (canceled)

30. A method as claimed in claim 1, wherein the subject is a human.

31-32. (canceled)

33. A method as claimed in claim 1, wherein one or more of the levels of the N endogenous metabolites are measured by spectroscopic techniques used in conjunction with a chemometric method, wherein the chemometric method is principal component analysis (PCA), partial least squares projections to latent structures (PLS), orthogonal PLS (OPLS), PLS discriminant analysis (PLS-DA) or orthogonal PLS-DA (OPLS-DA).

34-37. (canceled)

38. A method as claimed in claim 1, wherein the increases/decreases in the levels of the endogenous metabolites (between the subject and the control samples or between the samples taken at different time intervals from the subject) are significant increases/decreases.

39-53. (canceled)

54. A method as claimed in claim 2, wherein the biological sample is blood or synovial fluid.

55. A method as claimed in claim 2, wherein the subject is a human.

56. A method as claimed in claim 2, wherein one or more of the levels of the N endogenous metabolites are measured by spectroscopic techniques used in conjunction with a chemometric method, wherein the chemometric method is principal component analysis (PCA), partial least squares projections to latent structures (PLS), orthogonal PLS (OPLS), PLS discriminant analysis (PLS-DA) or orthogonal PLS-DA (OPLS-DA).

57. A method as claimed in claim 2, wherein the increases/decreases in the levels of the endogenous metabolites (between the subject and the control samples or between the samples taken at different time intervals from the subject) are significant increases/decreases.

58. A method as claimed in claim 21, wherein the biological sample is blood or synovial fluid.

59. A method as claimed in claim 21, wherein the subject is a human.

60. A method as claimed in claim 21, wherein one or more of the levels of the N endogenous metabolites are measured by spectroscopic techniques used in conjunction with a chemometric method, wherein the chemometric method is principal component analysis (PCA), partial least squares projections to latent structures (PLS), orthogonal PLS (OPLS), PLS discriminant analysis (PLS-DA) or orthogonal PLS-DA (OPLS-DA).

61. A method as claimed in claim 21, wherein the increases/decreases in the levels of the endogenous metabolites (between the subject and the control samples or between the samples taken at different time intervals from the subject) are significant increases/decreases.

62. A method as claimed in claim 22, wherein the biological sample is blood or synovial fluid.

63. A method as claimed in claim 22, wherein the subject is a human.

64. A method as claimed in claim 22, wherein one or more of the levels of the N endogenous metabolites are measured by spectroscopic techniques used in conjunction with a chemometric method, wherein the chemometric method is principal component analysis (PCA), partial least squares projections to latent structures (PLS), orthogonal PLS (OPLS), PLS discriminant analysis (PLS-DA) or orthogonal PLS-DA (OPLS-DA).

65. A method as claimed in claim 22, wherein the increases/decreases in the levels of the endogenous metabolites (between the subject and the control samples or between the samples taken at different time intervals from the subject) are significant increases/decreases.
